(19) United States
(12) Patent Application Publication
(10) Pub. No.: US 2023/0018666 A1
(43) Pub. Date: Jan. 19, 2023

(54) METHOD FOR PREPARING DEUTERATED AROMATIC COMPOUND, AND DEUTERATED REACTIVE COMPOSITION

(71) Applicant: LG CHEM, LTD., Seoul (KR)

(72) Inventors: Seungyeon Hwang, Daejeon (KR); Dongmin Jeong, Daejeon (KR); Dai Seung Choi, Daejeon (KR); Bethy Kim, Daejeon (KR)

(21) Appl. No.: 17/779,920

(22) PCT Filed: Aug. 27, 2021
(86) PCT No.: PCT/KR2021/011541

§ 371 Date: May 25, 2022

(30) Foreign Application Priority Data
Aug. 27, 2020 (KR) 10-2020-0108192
Dec. 18, 2020 (KR) 10-2020-0178795

(51) Int. Cl. C07D 307/91; C07D 333/76; C07D 209/88; C07D 487/04; C07D 495/04; C07D 493/04; C07D 251/22; C07D 409/08; C07D 405/08

(52) U.S. Cl. CPC C07D 307/91; C07D 333/76; C07D 209/88; C07D 487/04; C07D 495/04; C07D 493/04; C07D 251/22; C07D 409/08; C07D 405/08

(57) ABSTRACT

The present specification relates to a method for producing a deuterated aromatic compound and a deuterated reaction composition.

The present application is a National Phase entry pursuant to 35 U.S.C. § 371 of International Application No. PCT/KR2021/011541 filed on Aug. 27, 2021, and claims priority to and the benefit of Korean Patent Application Nos. 10-2020-0108192 and 10-2020-0178795 filed in the Korean Intellectual Property Office on Aug. 27, 2020 and Dec. 18, 2020, respectively, the entire contents of which are incorporated herein by reference.

### FIELD

The present specification relates to a method for producing a deuterated aromatic compound and a deuterated reaction composition.

### BACKGROUND

Compounds including deuterium are used for various purposes. For example, compounds including deuterium may be frequently used not only as labeling compounds for elucidating the mechanism of a chemical reaction or elucidating a material metabolism, but also for drugs, pesticides, organic EL materials and other purposes.

A method of deuterium substitution of an aromatic compound is known in order to improve the lifespan of an organic light emitting device (OLED) material. The principle of such an effect is that while the LUMO energy of C-D bond is lower than that of C—H bond during deuterium substitution, the lifetime characteristics of the OLED material are improved.

When a deuterated reaction was performed on one or more aromatic compounds using an existing heterogeneous catalytic reaction, there was a problem in that by-products due to side reactions continued to be generated. The by-products are caused by a hydrogenation reaction generated by hydrogen gas, and in order to remove the by-products, attempts have also been made to increase the purity through a purification process after the reaction, but it was difficult to obtain high purity because there was no difference in melting point and solubility from existing materials. When the reaction is performed without hydrogen gas in order to alleviate the problem, the reaction needs to be performed at a very high temperature (about 220° C. or higher), which may pose a safety problem.

### SUMMARY

The present specification has been made in an effort to provide a method for producing a deuterated aromatic compound and a deuterated reaction composition.

The present specification provides a method for producing a deuterated aromatic compound, the method including: performing a deuterated reaction of an aromatic compound including one or more aromatic rings using a solution including the aromatic compound, heavy water (D₂O), an organic compound which can be hydrolyzed by the heavy water, and an organic solvent.

Further, the present specification provides a deuterated reaction composition including an aromatic compound including one or more aromatic rings, heavy water (D₂O), an organic compound which can be hydrolyzed by the heavy water, and an organic solvent.

In addition, the present specification provides a deuterated aromatic compound prepared by the above-described method.

Furthermore, the present specification provides an electronic device including the deuterated aromatic compound.

A production method of a first exemplary embodiment according to the present specification has an advantage in that impurities due to hydrogen gas are not generated.

A production method of a second exemplary embodiment according to the present specification has an advantage in that the deuterium substitution rate is high.

A production method of a third exemplary embodiment according to the present specification has an advantage in that the purity of an obtained compound is high.

A production method of a fourth exemplary embodiment according to the present specification enables a deuterated reaction under a lower pressure.

A production method of a fifth exemplary embodiment according to the present specification enables a deuterated reaction at a lower temperature.

### DETAILED DESCRIPTION

Hereinafter, the present specification will be described in detail.

The present specification provides a method for producing a deuterated aromatic compound, the method including: performing a deuterated reaction of an aromatic compound including one or more aromatic rings using a solution including the aromatic compound, heavy water (D₂O), an organic compound which can be hydrolyzed by the heavy water, and an organic solvent.

The method for producing a deuterated aromatic compound of the present specification is characterized in that there is no hydrogen supply step.

In the related art, hydrogen gas was supplied in order to activate a metal catalyst, which is a heterogeneous catalyst added to produce a deuterated aromatic compound. When a deuterated reaction is performed by supplying hydrogen, a hydrogenation reaction is performed by hydrogen gas and thus by-products are generated by a side reaction.

In order to remove the generated by-products, a process of increasing the purity through a purification process after the reaction is required, and even though the purification process as described above is performed, the by-products have no difference in melting point and solubility from a target material, so that it is difficult to produce the deuterated aromatic compound with high purity.

The method for producing a deuterated aromatic compound of the present specification has an advantage in that impurities due to hydrogen gas are not generated because a metal catalyst and hydrogen gas for activating the metal catalyst need not be supplied due to the use of an organic compound which can be hydrolyzed by heavy water instead of the metal catalyst which is a heterogeneous catalyst.

Meanwhile, when a metal catalyst is used during the deuterated reaction, the metal catalyst reacts with a reactive group of a compound to be deuterated, that is, a halogen group, a hydroxyl group, and the like, so that in a deuterated reaction using a metal catalyst, the compound to be deuterated has no choice but to be limited to a compound having no reactive group capable of reacting with the metal catalyst, or a reactive group which has low reactivity.

Since an organic compound which can be hydrolyzed by heavy water is used instead of a metal catalyst, which is a heterogeneous catalyst, in the method for producing a deuterated aromatic compound of the present specification a compound having a reactive group such as a halogen group and a hydroxyl group may also be selected as the compound to be deuterated. Specifically, after a compound, which is an intermediate having a reactive group such as a halogen group and a hydroxyl group, is deuterated, a reaction of substituting the reactive group with an additional aromatic substituent may be performed.

The production method according to the present specification has an advantage in that the deuterium substitution rate is high.

The production method according to the present specification has an advantage in that the purity of an obtained compound is high.

The production method according to the present specification enables a deuterated reaction under a lower pressure.

The production method according to the present specification enables a deuterated reaction at a lower temperature.

The method for producing a deuterated aromatic compound of the present specification includes: preparing a solution including an aromatic compound including one or more aromatic rings, heavy water (D₂O), an organic compound which can be hydrolyzed by the heavy water, and an organic solvent.

The solution including the aromatic compound including one or more aromatic rings, heavy water (D₂O), the organic compound which can be hydrolyzed by the heavy water, and the organic solvent may be introduced into a reactor.

Alternatively, the aromatic compound including one or more aromatic rings, heavy water (D₂O), an organic compound which can be hydrolyzed by the heavy water, and an organic solvent can be individually introduced into a reactor.

The organic compound which can be hydrolyzed by the heavy water is not particularly limited as long as the organic compound has a reactive group which can be decomposed by heavy water, and the organic compound may include, for example, at least one compound of the following Chemical Formulae 1 to 4.

R1-C(O)OC(O)—R2 [Chemical Formula 1]

R3-S(O₂)OS(O₂)—R4 [Chemical Formula 2]

R5-C(O)O—R6 [Chemical Formula 3]

R7-CONH—R8 [Chemical Formula 4]

In Chemical Formulae 1 to 4, R1 to R8 are the same as or different from each other, and are each independently a monovalent organic group.

In an exemplary embodiment of the present specification, R1 and R2 may be the same substituent.

In an exemplary embodiment of the present specification, R3 and R4 may be the same substituent.

In an exemplary embodiment of the present specification, R5 and R6 may be the same substituent.

In an exemplary embodiment of the present specification, R7 and R8 may be the same substituent.

In an exemplary embodiment of the present specification, R1 to R8 are the same as or different from each other, and may be each independently an alkyl group which is unsubstituted or substituted with a halogen group; or an aryl group which is unsubstituted or substituted with a halogen group.

In an exemplary embodiment of the present specification, R1 to R8 are the same as or different from each other, and may be each independently an alkyl group having 1 to 30 carbon atoms, which is unsubstituted or substituted with a halogen group; or an aryl group having 6 to 50 carbon atoms, which is unsubstituted or substituted with a halogen group.

In an exemplary embodiment of the present specification, R1 to R8 are the same as or different from each other, and may be each independently an alkyl group having 1 to 10 carbon atoms, which is unsubstituted or substituted with a halogen group; or an aryl group having 6 to 20 carbon atoms, which is unsubstituted or substituted with a halogen group.

In an exemplary embodiment of the present specification, R1 to R8 are the same as or different from each other, and may be each independently an alkyl group having 1 to 10 carbon atoms, which is unsubstituted or substituted with a halogen group.

In an exemplary embodiment of the present specification, R1 to R8 are the same as or different from each other, and may be each independently an alkyl group having 1 to 5 carbon atoms, which is unsubstituted or substituted with a halogen group.

In an exemplary embodiment of the present specification, R1 to R8 are the same as or different from each other, and may be each independently a substituent of the following Chemical Formula 5 or 6.

—(CH₂)ₗ(CF₂)ₙ(CF₃)ₙ(CH₃)₁₋ₙ [Chemical Formula 5]

—C(H)ₐ((CH₂)ₗ(CF₂)ₙCF₃)₃₋ₐ [Chemical Formula 6]

In Chemical Formulae 5 and 6, 1 and m are each an integer from 0 to 10, and n and a are each 0 or 1.

In an exemplary embodiment of the present specification, R1 to R8 are the same as or different from each other, and may be each independently the substituent of Chemical Formula 5.

In an exemplary embodiment of the present specification, R1 to R8 are the same as or different from each other, and may be each independently —CF₃, —CH₂CH₃ or —CH₃.

In an exemplary embodiment of the present specification, the organic compound which can be hydrolyzed by the heavy water may include at least one of trifluoromethanesulfonic anhydride, trifluoroacetic anhydride, acetic anhydride and methanesulfonic anhydride.

In an exemplary embodiment of the present specification, the organic compound which can be hydrolyzed by the heavy water may include trifluoromethanesulfonic anhydride.

In an exemplary embodiment of the present specification, the organic compound which can be hydrolyzed by the heavy water may include trifluoroacetic anhydride.

In an exemplary embodiment of the present specification, the organic compound which can be hydrolyzed by the heavy water may include acetic anhydride.

In an exemplary embodiment of the present specification, the organic compound which can be hydrolyzed by the heavy water may include methanesulfonic anhydride.

In an exemplary embodiment of the present specification, the organic compound which can be hydrolyzed by the heavy water may include trifluoromethanesulfonic anhydride and trifluoroacetic anhydride.

In an exemplary embodiment of the present specification, the organic compound which can be hydrolyzed by the heavy water may include trifluoromethanesulfonic anhydride and acetic anhydride.

In an exemplary embodiment of the present specification, the organic compound which can be hydrolyzed by the heavy water may include methanesulfonic anhydride and trifluoroacetic anhydride.

In an exemplary embodiment of the present specification, the organic compound which can be hydrolyzed by the heavy water may include methanesulfonic anhydride and acetic anhydride.

In an exemplary embodiment of the present specification, the organic compound which can be hydrolyzed by the heavy water may include at least one of the compound of Chemical Formula 1 and the compound of Chemical Formula 2. When at least one of the compound of Chemical Formula 1 and the compound of Chemical Formula 2 is introduced into heavy water, hydrolysis with heavy water easily occurs even at room temperature.

In an exemplary embodiment of the present specification, the organic compound which can be hydrolyzed by the heavy water includes at least one of the compounds of Chemical Formula 1 and Chemical Formula 2, and may further include at least one of the compounds of Chemical Formula 3 and Chemical Formula 4. When the organic compound which can be hydrolyzed by the heavy water includes at least one of the compounds of Chemical Formula 1 and Chemical Formula 2, it is possible to control a temperature occurring due to a hydrolysis reaction which is an exothermic reaction by adding at least one of the compounds of Chemical Formula 3 and Chemical Formula 4 having a relatively slow hydrolysis reaction.

In an exemplary embodiment of the present specification, when the organic compound which can be hydrolyzed by the heavy water includes at least one of the compounds of Chemical Formula 3 and Chemical Formula 4, the organic compound may further include at least one of the compounds of Chemical Formula 1 and Chemical Formula 2. The hydrolysis reaction may be accelerated by adding at least one of the compounds of Chemical Formulas 1 and Chemical Formula 2, in which the hydrolysis reaction is relatively easily occurs.

In the organic compound which can be hydrolyzed by the heavy water, a weight ratio of at least one of the compounds of Chemical Formula 3 and Chemical Formula 4 to at least one of the compounds of Chemical Formula 1 and Chemical Formula 2 may be 100:0 to 0:100, 99:1 to 0:100, 90:10 to 0:100, 80:20 to 0:100, 70:30 to 0:100, 60:40 to 0:100, 50:50 to 0:100, 40:60 to 0:100, 30:70 to 0:100, 20:80 to 0:100, or 10:90 to 0:100.

According to an exemplary embodiment of the present specification, a content of the organic compound which can be hydrolyzed by the heavy water may be 1 wt % or more and 70 wt % or less, based on the total mass of the remaining compositions, excluding the aromatic compound in the above composition. In this case, there is an advantage in that it is possible to increase the affinity between the aromatic compound and heavy water which are immiscible with each other and a deuterium substitution reactivity is enhanced.

According to an exemplary embodiment of the present specification, the solution includes an organic solvent.

When the organic solvent is not used, in the case where a certain concentration or more of a hydrolyzed organic compound having deuterium by the hydrolysis reaction of a hydrolyzable organic compound is produced, the hydrolyzed organic compound having deuterium causes heavy water and an aromatic compound which is a target material to be mixed with each other, so that the deuterium substitution reaction is likely to occur.

However, since the organic compound hydrolyzed by heavy water itself is a superacid, an increase in the concentration of the hydrolyzed organic compound tends to cause a side reaction, thereby lowering the purity. In addition, it may also be dangerous in terms of stability to handle a solution containing a large amount of hydrolyzed organic compounds during the work-up process after the reaction.

In contrast, compared to the deuterated reaction without an organic solvent, when an organic solvent is used together, the amount of an organic compound which can be hydrolyzed by heavy water used may be reduced by about 30 to 90%, so that the purity may be increased and the stability may be improved.

In this case, the organic solvent which can be used in the reaction needs to be able to dissolve all the reactants and reaction products under the reaction conditions.

When the organic solvent is not used, the concentration of deuterium-substituted trifluoromethanesulfonic acid formed by the hydrolysis reaction of trifluoromethanesulfonic anhydride added as the organic compound which can be hydrolyzed by heavy water is increased, so that the deuterium substitution reaction is likely to occur.

However, since the trifluoromethanesulfonic acid itself is a superacid, an increase in the concentration of the trifluoromethanesulfonic acid tends to cause a side reaction, thereby lowering the purity. Furthermore, it may also be dangerous in terms of stability to handle a solution containing a large amount of trifluoromethanesulfonic acid during the work-up process after the reaction.

In contrast, when the organic solvent is used together, the amount of trifluoromethane sulfonic anhydride used may be reduced by about 30 to 90% compared to the existing amount, so that the purity may be increased and the stability may be improved.

The organic solvent may be selected from the group consisting of a hydrocarbon chain which is unsubstituted or substituted with a halogen group; an aliphatic hydrocarbon ring which is unsubstituted or substituted with an alkyl group; an aromatic hydrocarbon ring which is unsubstituted or substituted with an alkyl group; a straight-chained or branched heterochain; a substituted or unsubstituted aliphatic hetero ring; and a substituted or unsubstituted aromatic hetero ring. Specifically, the organic solvent includes at least one of an oxygen element and a sulfur atom, and is selected from the group consisting of a substituted or unsubstituted hetero ring; a substituted or unsubstituted alkyl acetate; alkyl ketone; alkyl sulfoxide; a lactone having 4 to 10 carbon atoms; alkylamide; a glycol having 4 to 10 carbon atoms; dioxane; an acetic acid which is unsubstituted or substituted with alkoxy.

For the deuterium substitution reaction to occur frequently, heavy water which is a supply source of deuterium and an aromatic compound which is to be substituted with deuterium need to be in one phase. However, heavy water and an aromatic compound which is a target material basically have the property of not being mixed well.

When the hydrolyzed organic compound is produced at a certain level or more, both heavy water and the aromatic compound are dissolved by the hydrolyzed organic compound, and a deuterium substitution reaction occurs. For example, when trifluoromethanesulfonic acid, which is a superacid, is produced in a certain amount or more by hydrolysis, both heavy water and an aromatic compound are dissolved by the trifluoromethanesulfonic acid, and a deuterium substitution reaction occurs.

In order to dissolve all the materials added and produced by the deuterium substitution reaction, the organic solvent needs to be well mixed with heavy water and also needs to be able to dissolve the aromatic compounds to some extent. Since the organic solvent needs to be polar to some degree in order to have the above properties, the organic solvent may include an element having high electronegativity, which is a property of withdrawing electrons. For example, the organic solvent may include an oxygen element and/or a sulfur element, which have/has a relatively good stability while having high electronegativity.

When the organic solvent has too much polarity, it is not possible to dissolve an aromatic compound which is relatively non-polar, so that it is appropriate for the polarity of the organic solvent to be between that of heavy water and that of the aromatic compound. When the organic solvent has a cyclic form, the organic solvent has a slight polarity compared to the case where the organic solvent is not cyclic, so that the miscibility is improved.

The organic solvent may be selected from the group consisting of ethyl acetate, acetone, cyclohexanone, methyl ethyl ketone, tetrahydrofuran, tetrahydropyran, cyclopentanone, 1,2-dioxane, 1,3-dioxane, 1,4-dioxane, N,N-dimethylformamide (DMF), dimethyl sulfoxide (DMSO), 1,2-dimethoxyethane, diglyme, γ-butyrolactone, γ-valerolactone, methyl ethyl diglycol (MEDG), propylene glycol methyl ether (PGME), propylene glycol methyl ether acetate (PGMEA), ethyl lactate, cyclohexane, methylcyclohexane, ethylcyclohexane, diethyl ether, 1,2-dimethoxyethane, decalin, hexane, heptane, toluene, xylene, 1,3,5-trimethylbenzene, dichloromethane, 1,2-dichloroethane, 1,1,2,2-tetrachloroethane, tetrachloroethylene and 2-methoxyacetic acid.

When the content of the organic solvent is too large, the deuterium substitution rate decreases, and conversely, when the content of the organic solvent is too small, the reactants cannot be dissolved well, and thus the deuterium substitution rate decreases. Preferably, the mass ratio of the organic solvent may be 4-fold to 40-fold, specifically 4-fold to 16-fold, based on the mass of the aromatic compound.

According to an exemplary embodiment of the present specification, the solution is characterized by the fact that the solution does not contain a metal catalyst. The organic compound which can be hydrolyzed by heavy water plays the role of the metal catalyst. Through this, problems caused by using a metal catalyst, for example, the fact that hydrogen gas needs to be supplied, the fact that impurities due to hydrogen gas need to be removed, the fact that the process equipment capable of maintaining and withstanding a high reaction temperature and a high pressure needs to be provided, and the like, are solved.

According to an exemplary embodiment of the present specification, the solution includes heavy water.

According to an exemplary embodiment of the present specification, the content of heavy water may be 0.1-fold or more and 30-fold or less, of the weight of the aromatic compound. In this case, there is an advantage in that deuterium can be efficiently substituted from heavy water.

According to an exemplary embodiment of the present specification, the solution may include an additional deuterium source as well as heavy water. The deuterium source may be a deuterated aromatic solvent, for example, benzene-d6, toluene-d8, and the like.

According to an exemplary embodiment of the present specification, the content of the additional deuterium source may be 0.1-fold or more and 30-fold or less, of the weight of the aromatic compound. In this case, there is an advantage in that the reactivity can be enhanced and the heat generation during the reaction can be reduced.

In an exemplary embodiment of the present specification, the aromatic compound is an aromatic compound including one or more aromatic rings, and specifically, is an aromatic compound including 1 to 30 aromatic rings. In this case, the meaning of having one or more aromatic rings means that there may be one or more aromatic rings of a monocyclic ring, a polycyclic ring, or a combination thereof, or there may be one or more aromatic rings (for example, a benzene ring) which are a basic unit. For example, the carbazole ring may mean one aromatic ring, or may mean that two benzene rings are linked or three rings including a benzene ring are fused, based on a ring fused with a benzene ring which is a basic unit.

According to an exemplary embodiment of the present specification, the content of the aromatic compound may be 3 wt % or more and 50 wt % or less, based on the total weight of the solution.

In an exemplary embodiment of the present specification, the aromatic ring may be a substituted or unsubstituted, monocyclic or polycyclic hydrocarbon aromatic rings, or a substituted or unsubstituted, monocyclic or polycyclic heteroaromatic ring. For example, the aromatic ring may be a substituted or unsubstituted benzene ring, a substituted or unsubstituted naphthalene ring, a substituted or unsubstituted anthracene ring, a substituted or unsubstituted dibenzofuran, a substituted or unsubstituted dibenzothiophene, a substituted or unsubstituted carbazole, and the like.

In an exemplary embodiment of the present specification, the aromatic compound may be a heteroaromatic compound, and the heteroaromatic compound may be a carbazole-based compound, a dibenzofuran-based compound, a dibenzothiophene-based compound, a pyridine-based compound, a pyrimidine-based compound, or a triazine-based compound. The heteroaromatic compound means a compound including a heterogeneous element such as O, S, N, Si, P, and Se in addition to the carbon constituting a backbone, the hydrogen substituted with the corresponding backbone may be substituted with another substituent, and in this case, the type of substituent is not particularly limited.

In an exemplary embodiment of the present specification, the heteroaromatic compound is a compound including at least one of O, S and N and including a substituted or unsubstituted heteroaromatic ring.

In an exemplary embodiment of the present specification, the heteroaromatic compound is a compound including a heteroaromatic ring including a substituted or unsubstituted oxygen element.

In an exemplary embodiment of the present specification, the heteroaromatic compound is a compound including a heteroaromatic ring including a substituted or unsubstituted nitrogen element.

In an exemplary embodiment of the present specification, the heteroaromatic compound is a compound including a heteroaromatic ring including a substituted or unsubstituted sulfur element.

In an exemplary embodiment of the present specification, the heteroaromatic compound may be a carbazole-based compound, and specifically, may be a substituted or unsubstituted carbazole; or a substituted or unsubstituted carbazole having an additional ring to which an adjacent group is bonded.

The carbazole having an additional ring to which an adjacent group is bonded may be a substituted or unsubstituted benzocarbazole; a substituted or unsubstituted dibenzocarbazole; a substituted or unsubstituted furocarbazole; or a substituted or unsubstituted indolocarbazole.

In an exemplary embodiment of the present specification, the heteroaromatic compound may be a dibenzofuran-based compound, and specifically, may be a substituted or unsubstituted dibenzofuran; or a substituted or unsubstituted dibenzofuran having an additional ring to which an adjacent group is bonded.

In an exemplary embodiment of the present specification, the heteroaromatic compound may be a dibenzothiophene-based compound, and specifically, may be a substituted or unsubstituted dibenzothiophene; or a substituted or unsubstituted dibenzothiophene having an additional ring to which an adjacent group is bonded.

In an exemplary embodiment of the present specification, the heteroaromatic compound may be a substituted or unsubstituted indole; a substituted or unsubstituted benzofuran; a substituted or unsubstituted benzothiophene; a substituted or unsubstituted benzoxazole; a substituted or unsubstituted benzothiazole; a substituted or unsubstituted benzoimidazole; a substituted or unsubstituted anthraquinone; a substituted or unsubstituted xanthene; a substituted or unsubstituted thioxanthene; a substituted or unsubstituted pyridine; a substituted or unsubstituted pyrimidine; a substituted or unsubstituted triazine; or dihydroindolocarbazole.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, the term “substituted or unsubstituted” means being substituted with one or two or more substituents selected from the group consisting of a halogen group; a nitrile group; a nitro group; a hydroxyl group; an amine group; a silyl group; a boron group; an alkoxy group; an alkyl group; a cycloalkyl group; an aryl group; and a heterocyclic group, being substituted with a substituent to which two or more substituents among the above-exemplified substituents are linked, or having no substituent. For example, “the substituent to which two or more substituents are linked” may be a biphenyl group. That is, the biphenyl group may also be an aryl group, and may be interpreted as a substituent to which two phenyl groups are linked.

In the present specification, examples of a halogen group include fluorine (—F), chlorine (—Cl), bromine (—Br) or iodine (—I).

In the present specification, a silyl group may be represented by a chemical formula of —SiYₐY_{b}Y_{c}, and the Yₐ, Y_{b}, and Y_{c} may be each hydrogen; a substituted or unsubstituted alkyl group; or a substituted or unsubstituted aryl group. Specific examples of the silyl group include a trimethylsilyl group, a triethylsilyl group, a tert-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be represented by a chemical formula of —BY_{d}Yₑ, and the Y_{d} and Yₑ may be each hydrogen; a substituted or unsubstituted alkyl group; or a substituted or unsubstituted aryl group. Specific examples of the boron group include a dimethylboron group, a diethylboron group, a tert-butylmethylboron group, a diphenylboron group, a phenylboron group, and the like, but are not limited thereto.

In the present specification, the alkyl group may be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

In the present specification, the alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, and the like, but are not limited thereto.

Substituents including an alkyl group, an alkoxy group, and other alkyl group moieties described in the present specification include both a straight-chained form and a branched form.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to yet another exemplary embodiment, the number of carbon atoms of the cycloalkyl group is 3 to 6. Specific examples thereof include a cyclopropyl group, a cyclobutyl group, a cyclopentyl group, a cyclohexyl group, a cycloheptyl group, a cyclooctyl group, and the like, but are not limited thereto.

In the present specification, an aryl group is not particularly limited, but has preferably 6 to 60 carbon atoms, and may be a monocyclic aryl group or a polycyclic aryl group. According to an exemplary embodiment, the number of carbon atoms of the aryl group is 6 to 39. According to an exemplary embodiment, the number of carbon atoms of the aryl group is 6 to 30. Examples of the monocyclic aryl group include a phenyl group, a biphenyl group, a terphenyl group, a quarterphenyl group, and the like, but are not limited thereto. Examples of the polycyclic aryl group include a naphthyl group, an anthracenyl group, a phenanthrenyl group, a pyrenyl group, a perylenyl group, a triphenyl group, a chrysenyl group, a fluorenyl group, a triphenylenyl group, and the like, but are not limited thereto.

In the present specification, a fluorene group may be substituted, and two substituents may be bonded to each other to form a spiro structure.

When the fluorene group is substituted, the fluorene group may be a spirofluorene group such as

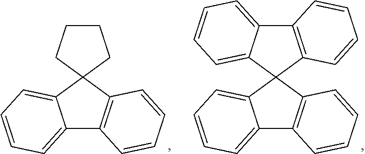

and a substituted fluorene group such as

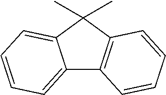

(a 9,9-dimethylfluorene group) and

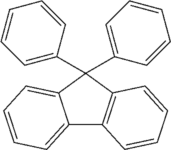 [SMILES: c1ccc(C2(c3ccccc3)c3ccccc3-c3ccccc32)cc1]

(a 9,9-diphenylfluorene group). However, the substituent is not limited thereto.

In the present specification, a heterocyclic group is a cyclic group including one or more of N, O, P, S, Si, and Se as a heteroatom, and the number of carbon atoms thereof is not particularly limited, but is preferably 2 to 60. According to an exemplary embodiment, the number of carbon atoms of the heterocyclic group is 2 to 36. Examples of the heterocyclic group include a pyridine group, a pyrrole group, a pyrimidine group, a quinoline group, a pyridazine group, a furan group, a thiophene group, an imidazole group, a pyrazole group, a dibenzofuran group, a dibenzothiophene group, a carbazole group, a benzocarbazole group, a benzonaphthofuran group, a benzonaphthothiophene group, an indenocarbazole group, an indolocarbazole group, and the like, but are not limited thereto.

In the present specification, the above-described description on the heterocyclic group may be applied to a heteroaryl group except for an aromatic heteroaryl group.

In the present specification, an amine group may be selected from the group consisting of —NH2; an alkylamine group; an N-alkylarylamine group; an arylamine group; an N-arylheteroarylamine group; an N-alkylheteroarylamine group; and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, an N-phenylnaphthylamine group, a ditolylamine group, an N-phenyltolylamine group, an N-phenylbiphenylamine group, an N-phenylnaphthylamine group, an N-biphenylnaphthylamine group, an N-naphthylfluorenylamine group, an N-phenylphenanthrenylamine group, an N-biphenylphenanthrenylamine group, an N-phenylfluorenylamine group, an N-phenyl terphenylamine group, an N-phenanthrenylfluorenylamine group, an N-biphenylfluorenylamine group, and the like, but are not limited thereto.

In the present specification, an N-alkylarylamine group means an amine group in which an alkyl group and an aryl group are substituted with N of the amine group.

In the present specification, an N-arylheteroarylamine group means an amine group in which an aryl group and a heteroaryl group are substituted with N of the amine group.

In the present specification, an N-alkylheteroarylamine group means an amine group in which an alkyl group and a heteroaryl group are substituted with N of the amine group.

In the present specification, an alkyl group, an aryl group, and a heteroaryl group in an alkylamine group; an N-alkylarylamine group; an arylamine group; an N-arylheteroarylamine group; an N-alkylheteroarylamine group, and a heteroarylamine group, are each the same as the above-described examples of the alkyl group, the aryl group, and the heteroaryl group.

In an exemplary embodiment of the present specification, the aromatic compound participating in the deuterated reaction may be any one of the following Chemical Formulae 7 to 10. By the deuterated reaction, at least one hydrogen of the selected compounds is substituted with deuterium.

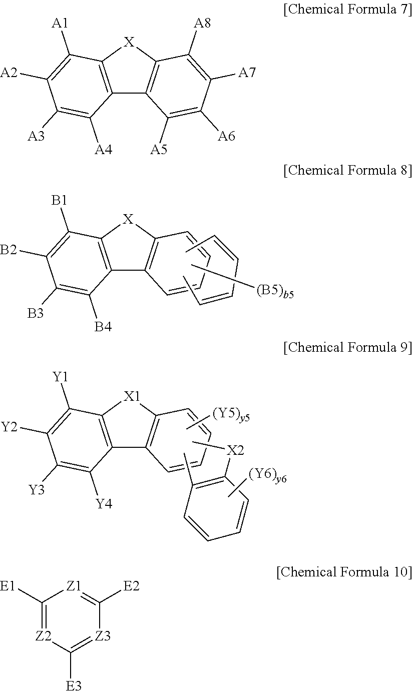

In Chemical Formulae 7 to 10,

X, X1 and X2 are each independently 0, S or NR, wherein

R is hydrogen; deuterium; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

A1 to A8 are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

B1 to B5 are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

E1 to E3 are each independently hydrogen; a leaving group; a substituted or unsubstituted alkyl group; a substituted or unsubstituted cycloalkyl group; a substituted or unsubstituted aryl group; or a substituted or unsubstituted heterocyclic group,

Y1 to Y6 are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

at least one of Z1 to Z3 is N, and the others are each independently CH or N,

b5 is an integer from 1 to 6, and when b5 is 2 or higher, B5's are the same as or different from each other,

y5 is 1 or 2, and when y5 is 2, Y5's are the same as or different from each other, and

y6 is an integer from 1 to 4, and when y6 is 2 or higher, Y6's are the same as or different from each other.

In an exemplary embodiment of the present specification, X is O.

In an exemplary embodiment of the present specification, X is S.

In an exemplary embodiment of the present specification, X is NR, and R is hydrogen; deuterium; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

In an exemplary embodiment of the present specification, X is NR, and R is hydrogen; deuterium; a substituted or unsubstituted aryl group; or a substituted or unsubstituted heterocyclic group.

In an exemplary embodiment of the present specification, at least one of A1 to A8 is a leaving group; a hydroxyl group; a substituted or unsubstituted amine group; or a cyano group, and the others are each independently hydrogen; a substituted or unsubstituted alkyl group; a substituted or unsubstituted cycloalkyl group; a substituted or unsubstituted aryl group; or a substituted or unsubstituted heterocyclic group.

In an exemplary embodiment of the present specification, at least one of B1 to B5 is a leaving group; a hydroxyl group; a substituted or unsubstituted amine group; or a cyano group, and the others are each independently hydrogen; a substituted or unsubstituted alkyl group; a substituted or unsubstituted cycloalkyl group; a substituted or unsubstituted aryl group; or a substituted or unsubstituted heterocyclic group.

In an exemplary embodiment of the present specification, at least one of Y1 to Y6 is a leaving group; a hydroxyl group; a substituted or unsubstituted amine group; or a cyano group, and the others are each independently hydrogen; a substituted or unsubstituted alkyl group; a substituted or unsubstituted cycloalkyl group; a substituted or unsubstituted aryl group; or a substituted or unsubstituted heterocyclic group.

In an exemplary embodiment of the present specification, any one of Z1 to Z3 is N, and the others are CH.

In an exemplary embodiment of the present specification, two of Z1 to Z3 are N, and the other is CH.

In an exemplary embodiment of the present specification, Z1 to Z3 are all N.

In an exemplary embodiment of the present specification, at least one of E1 to E3 is a leaving group, and the others are each independently hydrogen; a leaving group; a substituted or unsubstituted alkyl group; a substituted or unsubstituted cycloalkyl group; a substituted or unsubstituted aryl group; or a substituted or unsubstituted heterocyclic group.

In an exemplary embodiment of the present specification, the aromatic compound may be any one of the following structures.

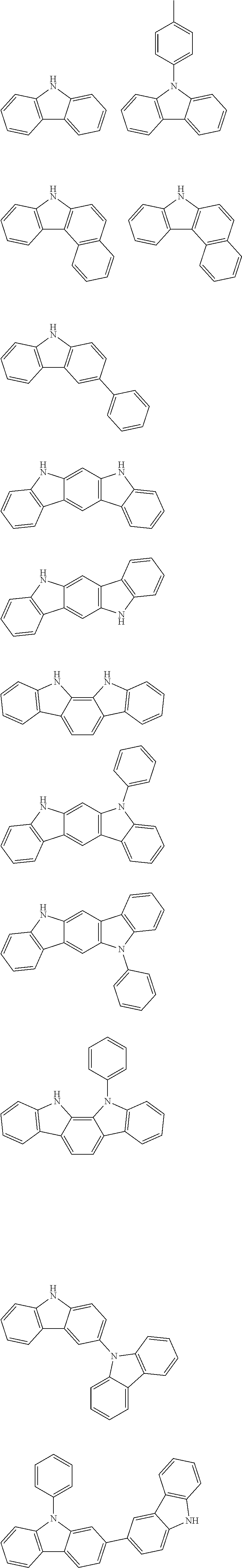

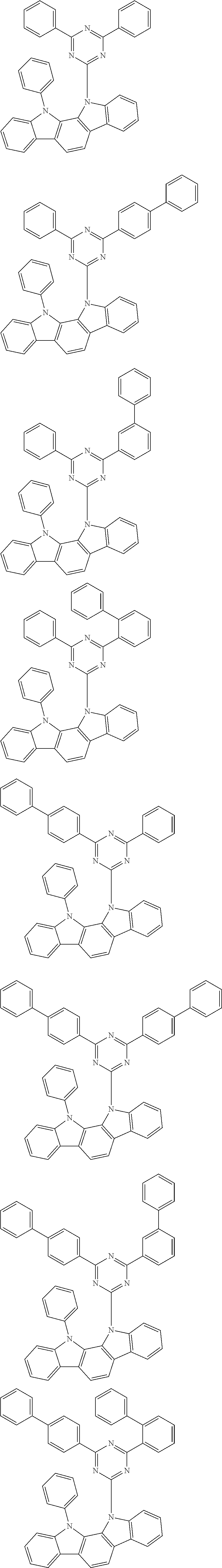

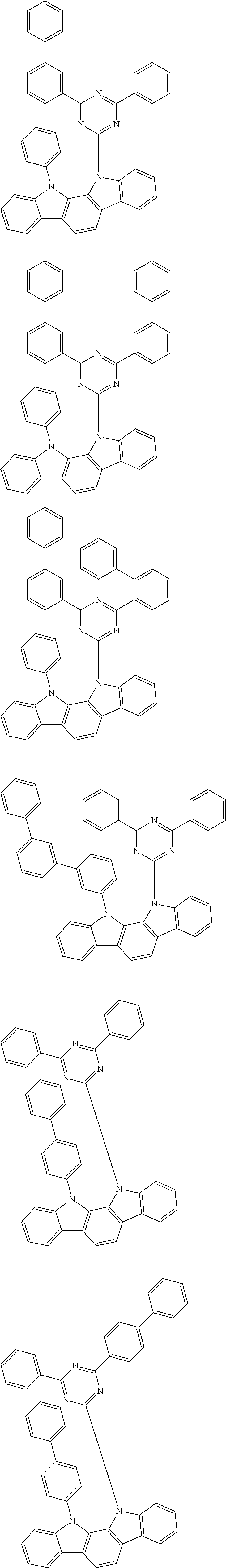

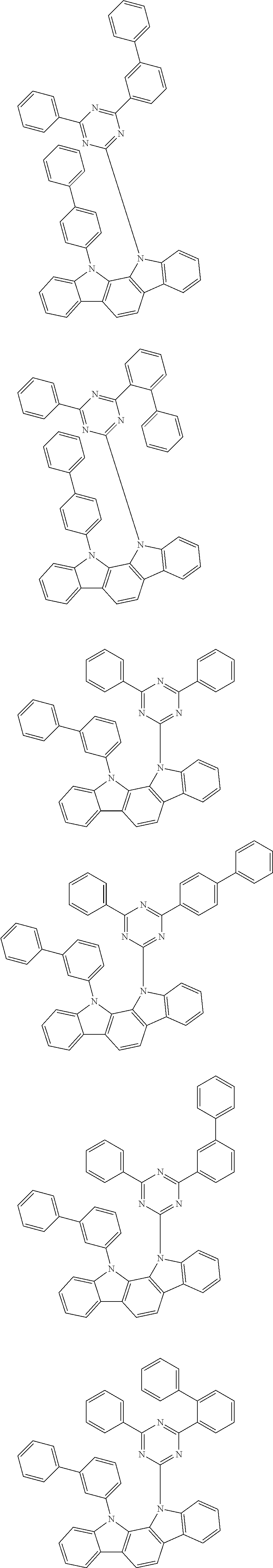

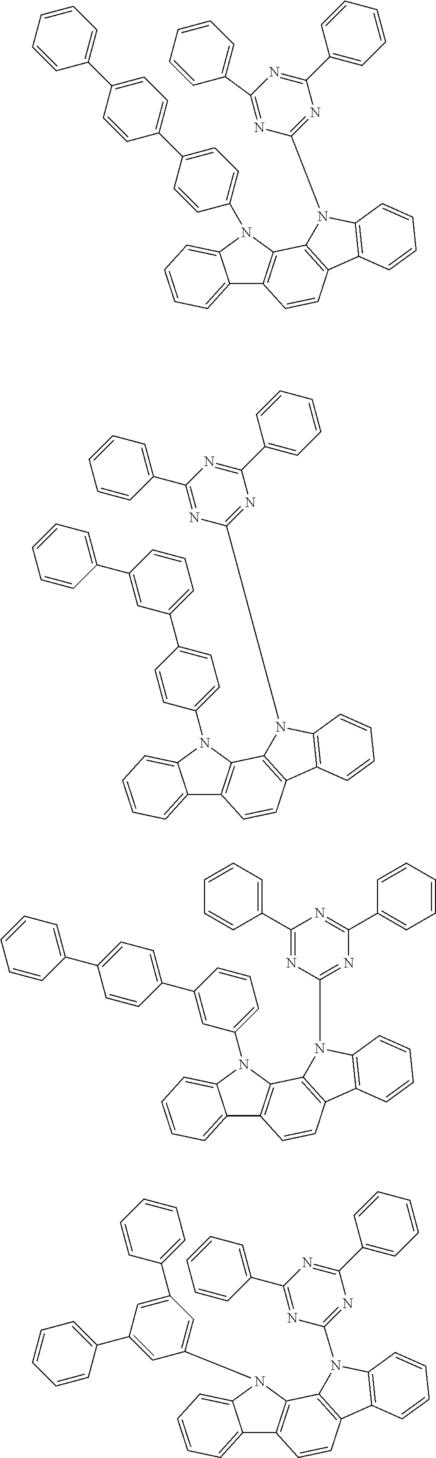

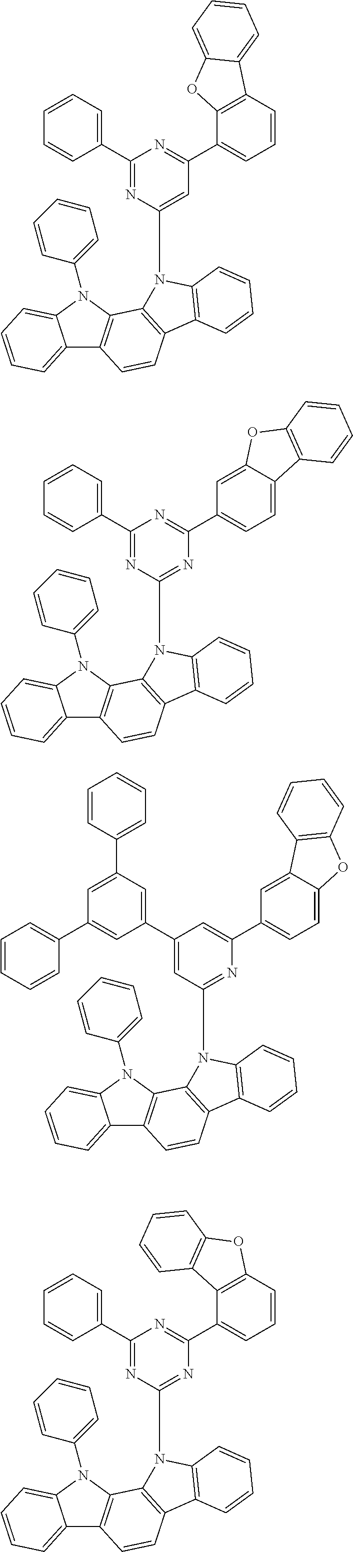

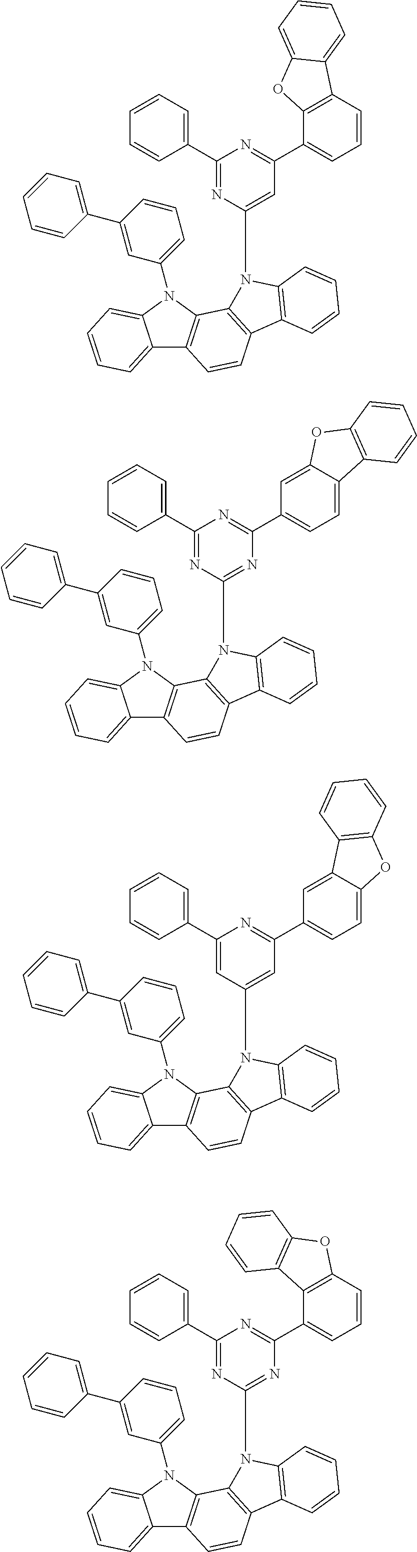 [SMILES: c1ccc(-c2cccc(-n3c4ccccc4c4ccc5c6ccccc6n(-c6cc(-c7cccc8c7oc7ccccc78)nc(-c7ccccc7)n6)c5c43)c2)cc1 ; c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1 ; c1ccc(-c2cccc(-n3c4ccccc4c4ccc5c6ccccc6n(-c6cc(-c7ccccc7)nc(-c7ccc8oc9ccccc9c8c7)c6)c5c43)c2)cc1 ; c1ccc(-c2cccc(-n3c4ccccc4c4ccc5c6ccccc6n(-c6nc(-c7ccccc7)nc(-c7cccc8oc9ccccc9c78)n6)c5c43)c2)cc1]

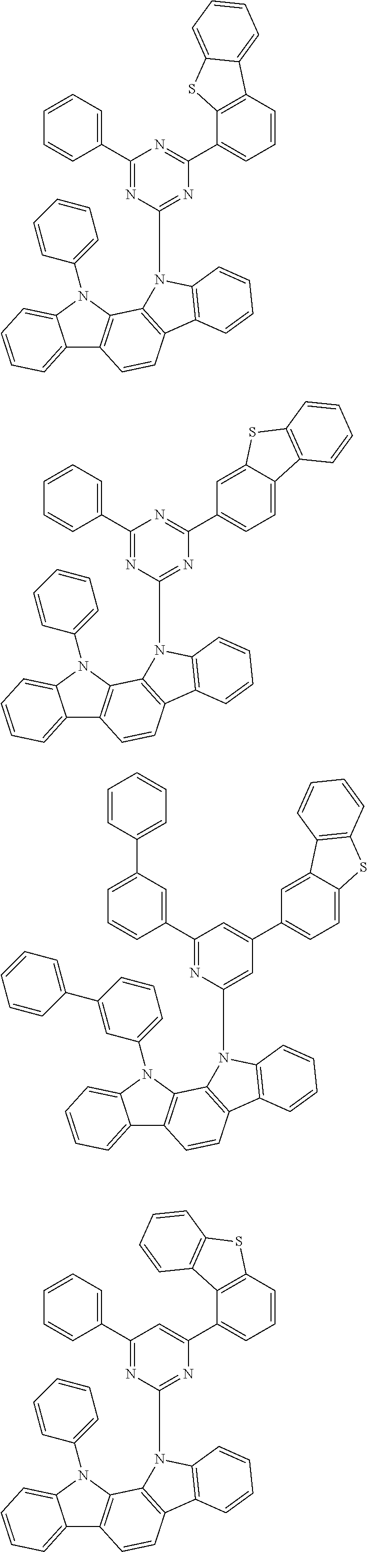

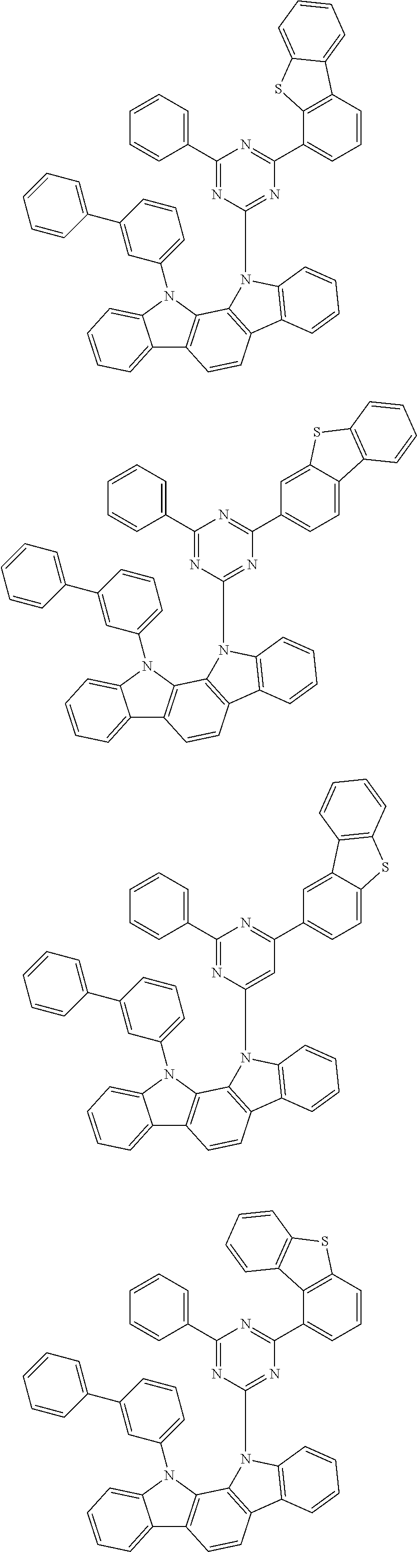 [SMILES: c1ccc(-c2cccc(-n3c4ccccc4c4ccc5c6ccccc6n(-c6nc(-c7ccccc7)nc(-c7cccc8c7sc7ccccc78)n6)c5c43)c2)cc1 ; c1ccc(-c2cccc(-n3c4ccccc4c4ccc5c6ccccc6n(-c6nc(-c7ccccc7)nc(-c7ccc8c(c7)sc7ccccc78)n6)c5c43)c2)cc1 ; c1ccc(-c2cccc(-n3c4ccccc4c4ccc5c6ccccc6n(-c6cc(-c7ccc8sc9ccccc9c8c7)nc(-c7ccccc7)n6)c5c43)c2)cc1 ; c1ccc(-c2cccc(-n3c4ccccc4c4ccc5c6ccccc6n(-c6nc(-c7ccccc7)nc(-c7cccc8sc9ccccc9c78)n6)c5c43)c2)cc1]

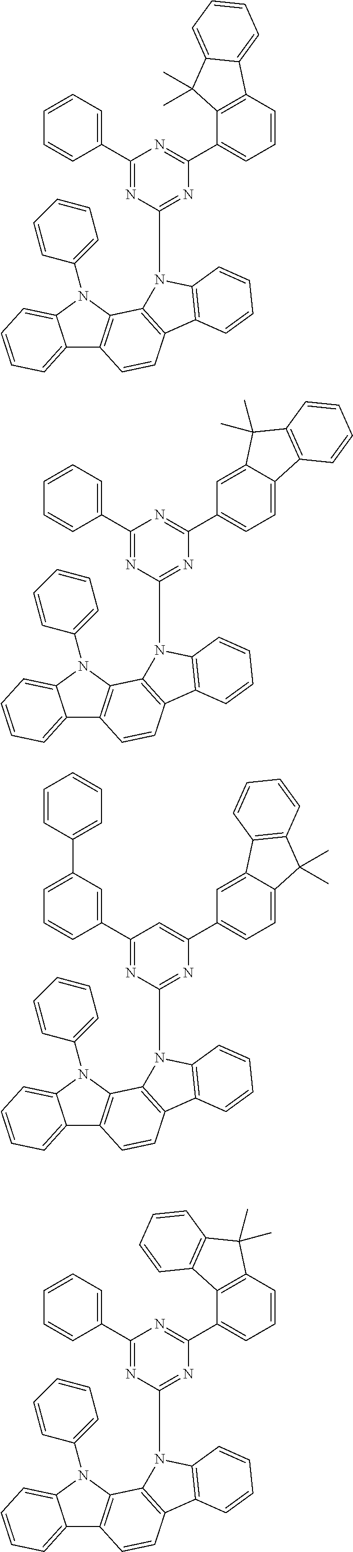

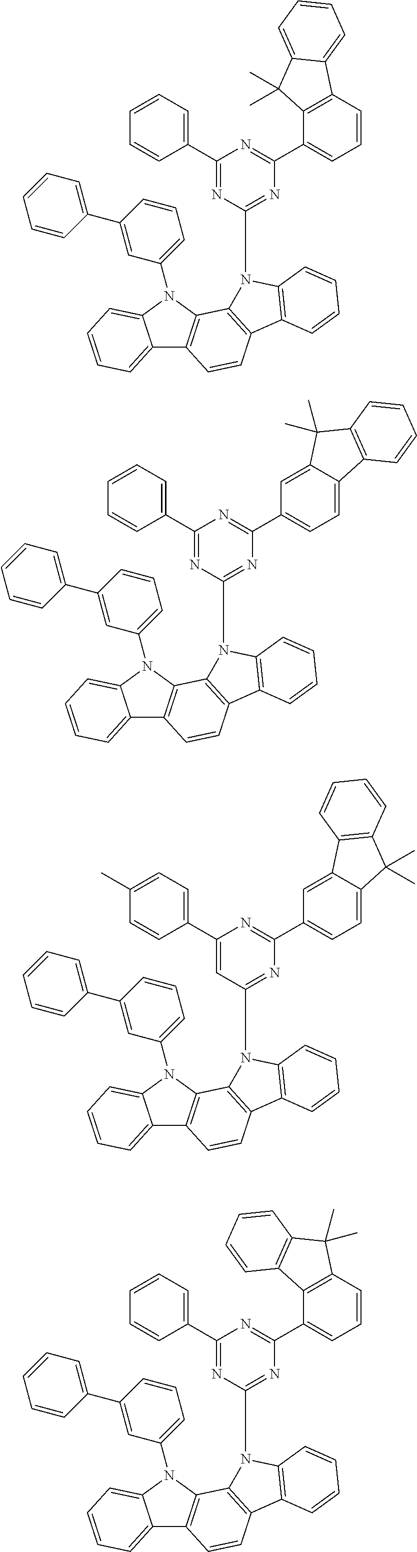 [SMILES: CC1(C)c2ccccc2-c2cccc(-c3nc(-c4ccccc4)nc(-n4c5ccccc5c5ccc6c7ccccc7n(-c7cccc(-c8ccccc8)c7)c6c54)n3)c21 ; CC1(C)c2ccccc2-c2ccc(-c3nc(-c4ccccc4)nc(-n4c5ccccc5c5ccc6c7ccccc7n(-c7cccc(-c8ccccc8)c7)c6c54)n3)cc21 ; Cc1ccc(-c2cc(-n3c4ccccc4c4ccc5c6ccccc6n(-c6cccc(-c7ccccc7)c6)c5c43)nc(-c3ccc4c(c3)-c3ccccc3C4(C)C)n2)cc1 ; CC1(C)c2ccccc2-c2c(-c3nc(-c4ccccc4)nc(-n4c5ccccc5c5ccc6c7ccccc7n(-c7cccc(-c8ccccc8)c7)c6c54)n3)cccc21]

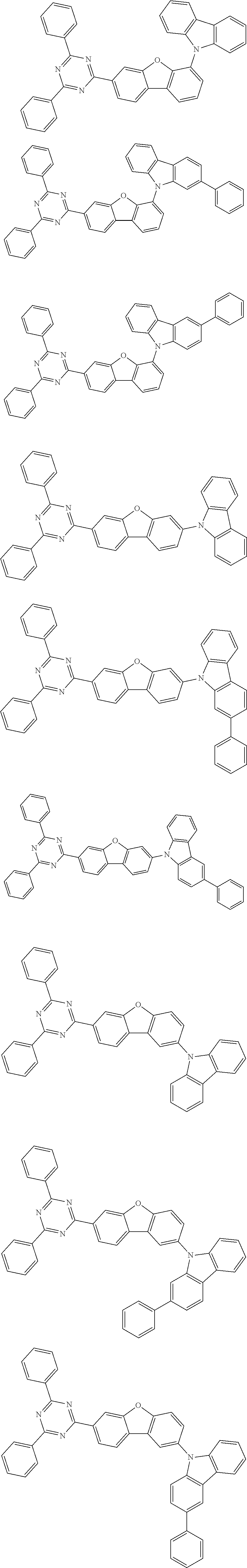

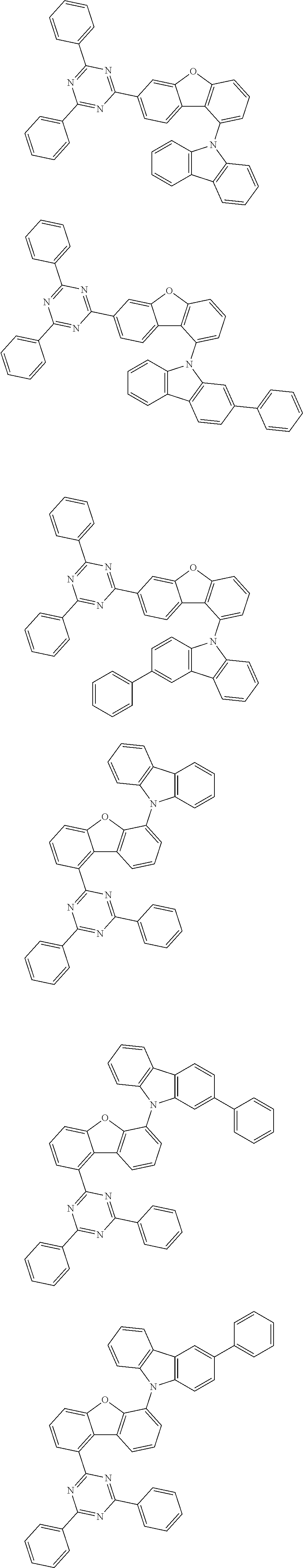

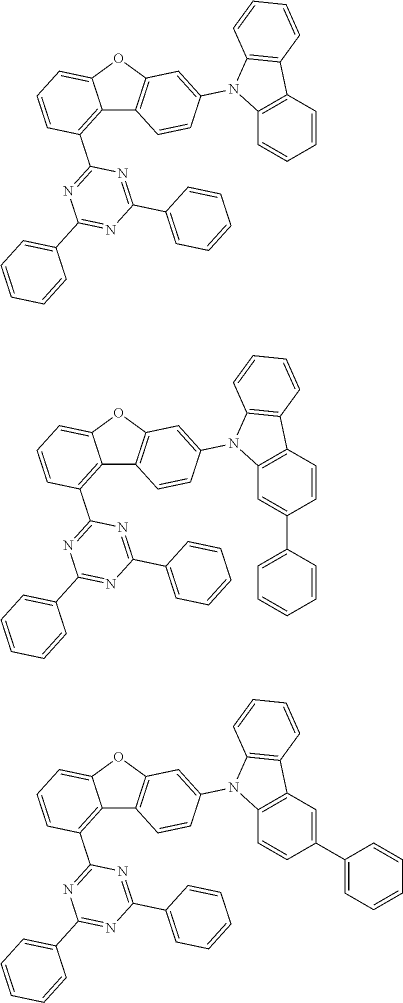 [SMILES: c1ccc(-c2nc(-c3ccccc3)nc(-c3cccc4oc5cc(-n6c7ccccc7c7ccccc76)ccc5c34)n2)cc1 ; c1ccc(-c2ccc3c4ccccc4n(-c4ccc5c(c4)oc4cccc(-c6nc(-c7ccccc7)nc(-c7ccccc7)n6)c45)c3c2)cc1 ; c1ccc(-c2ccc3c(c2)c2ccccc2n3-c2ccc3c(c2)oc2cccc(-c4nc(-c5ccccc5)nc(-c5ccccc5)n4)c23)cc1]

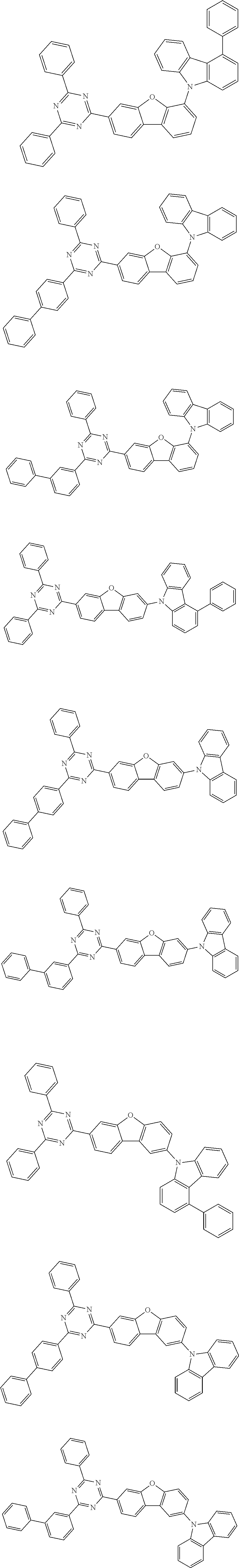

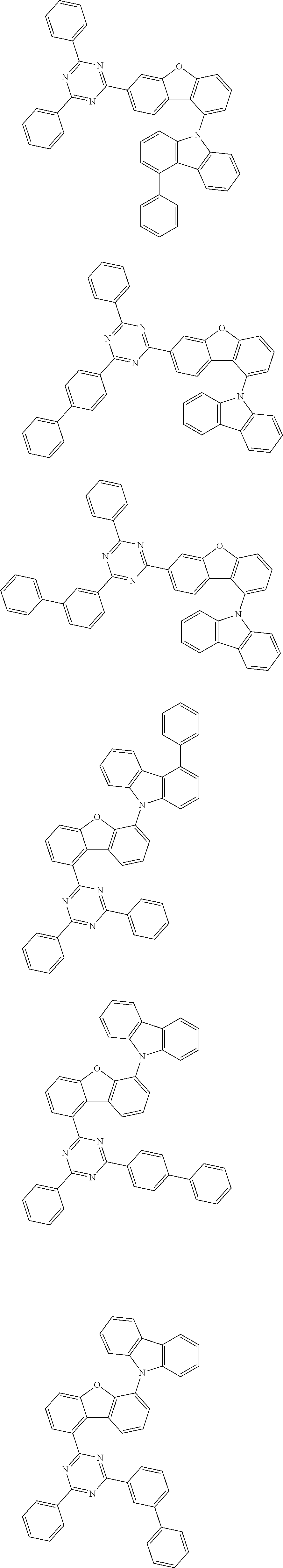

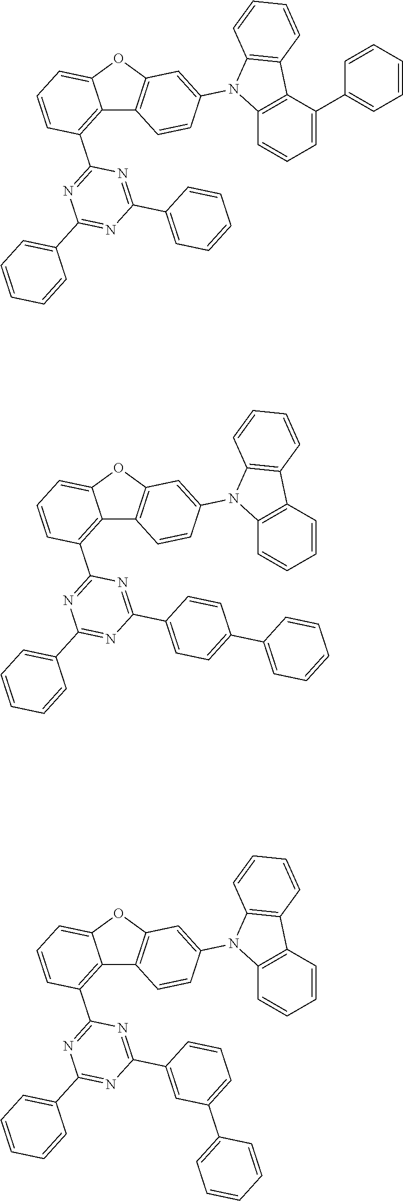 [SMILES: c1ccc(-c2nc(-c3ccccc3)nc(-c3cccc4oc5cc(-n6c7ccccc7c7c(-c8ccccc8)cccc76)ccc5c34)n2)cc1 ; c1ccc(-c2ccc(-c3nc(-c4ccccc4)nc(-c4cccc5oc6cc(-n7c8ccccc8c8ccccc87)ccc6c45)n3)cc2)cc1 ; c1ccc(-c2cccc(-c3nc(-c4ccccc4)nc(-c4cccc5oc6cc(-n7c8ccccc8c8ccccc87)ccc6c45)n3)c2)cc1]

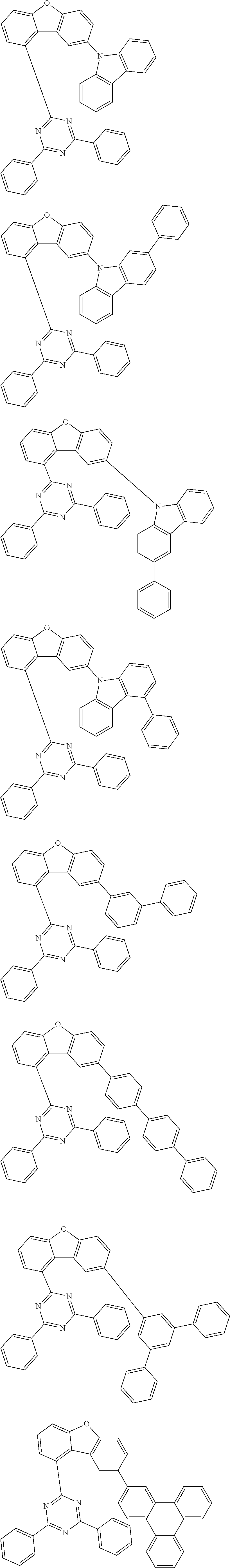

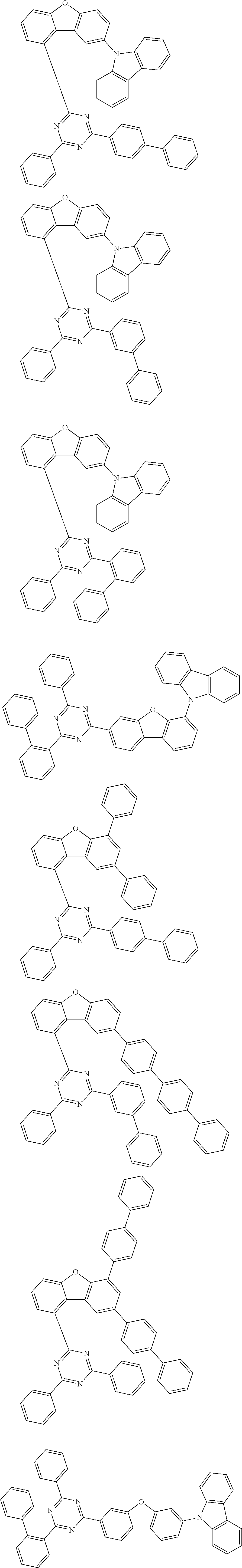

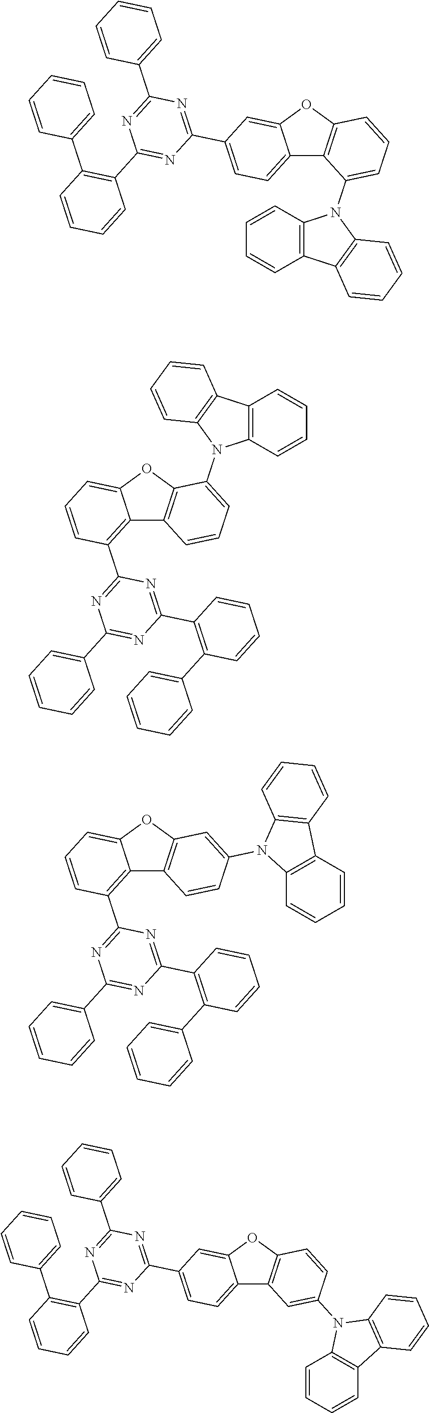 [SMILES: c1ccc(-c2nc(-c3ccc4c(c3)oc3cccc(-n5c6ccccc6c6ccccc65)c34)nc(-c3ccccc3-c3ccccc3)n2)cc1 ; c1ccc(-c2nc(-c3ccccc3-c3ccccc3)nc(-c3cccc4oc5c(-n6c7ccccc7c7ccccc76)cccc5c34)n2)cc1 ; c1ccc(-c2nc(-c3ccccc3-c3ccccc3)nc(-c3cccc4oc5cc(-n6c7ccccc7c7ccccc76)ccc5c34)n2)cc1 ; c1ccc(-c2nc(-c3ccc4c(c3)oc3ccc(-n5c6ccccc6c6ccccc65)cc34)nc(-c3ccccc3-c3ccccc3)n2)cc1]

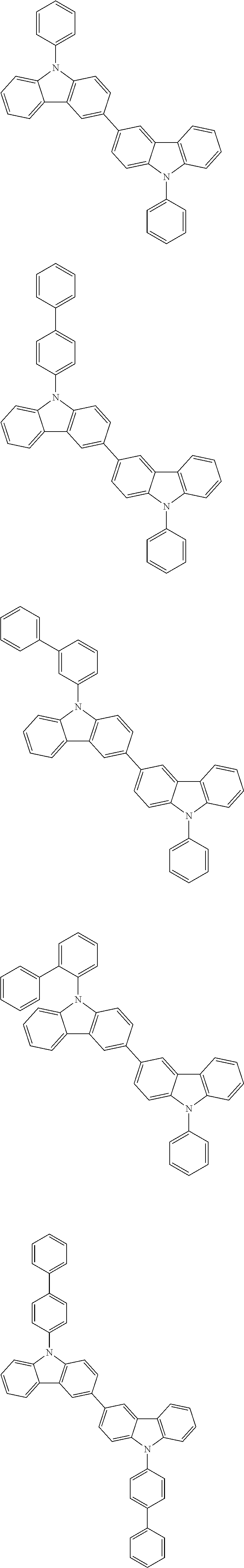 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

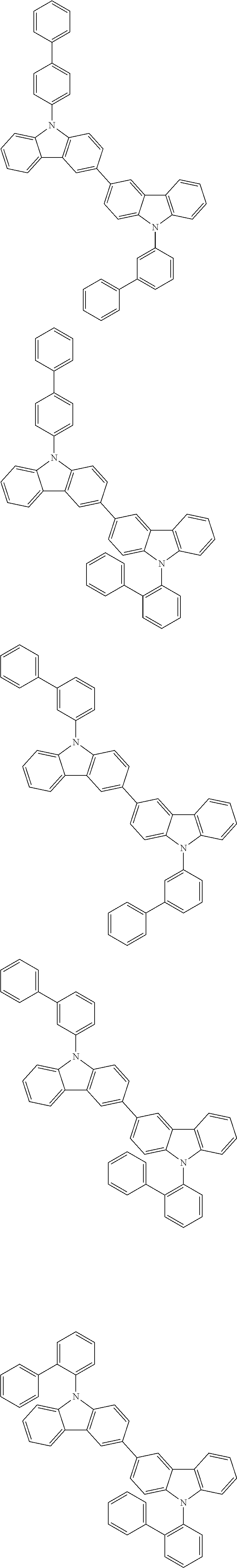

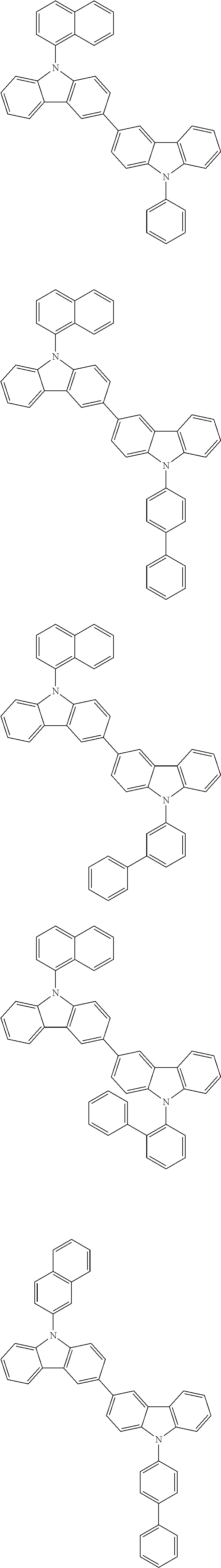

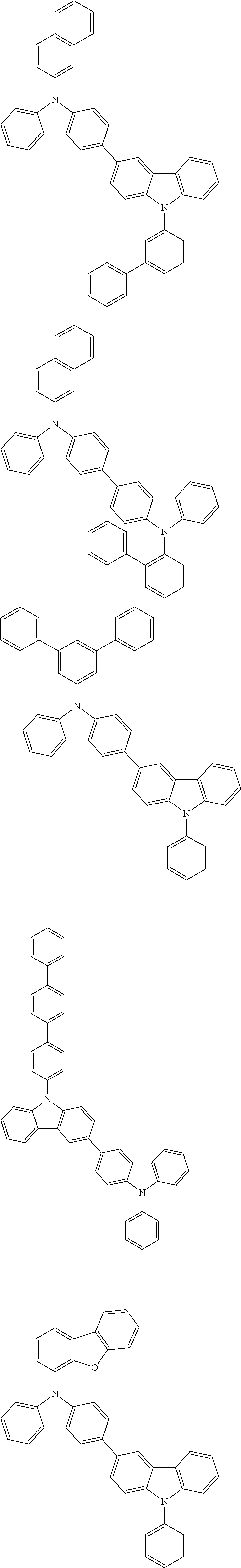

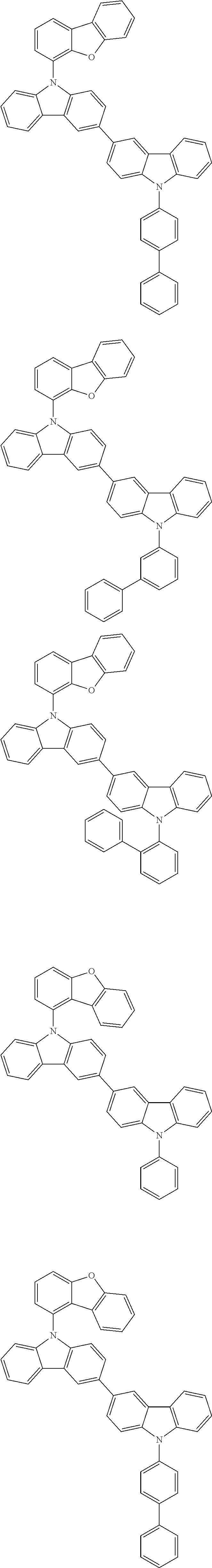

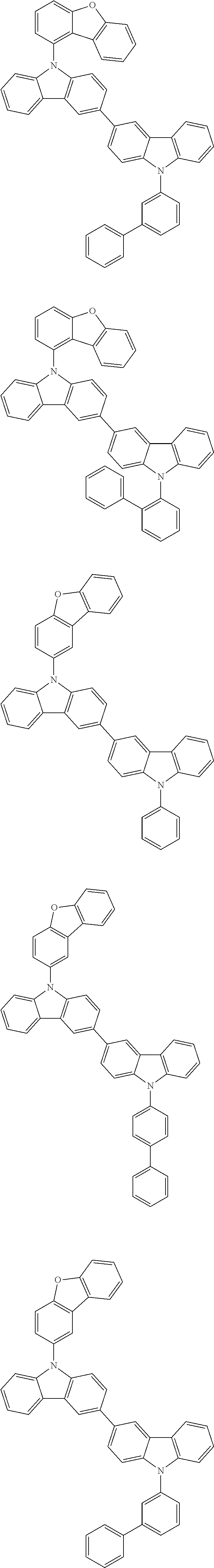 [SMILES: c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2ccccc2-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

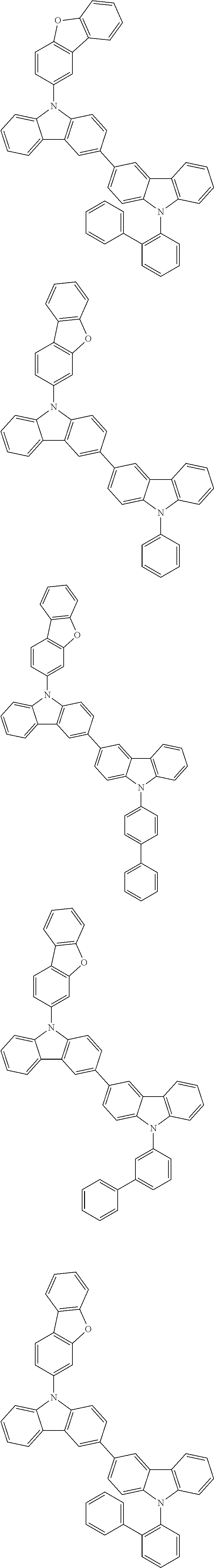 [SMILES: c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2ccccc2-n2c3ccccc3c3cc(-c4ccc5c(c4)c4ccccc4n5-c4ccc5c(c4)oc4ccccc45)ccc32)cc1]

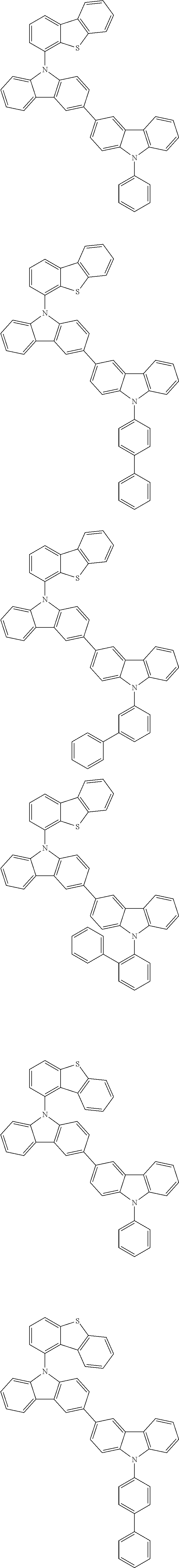

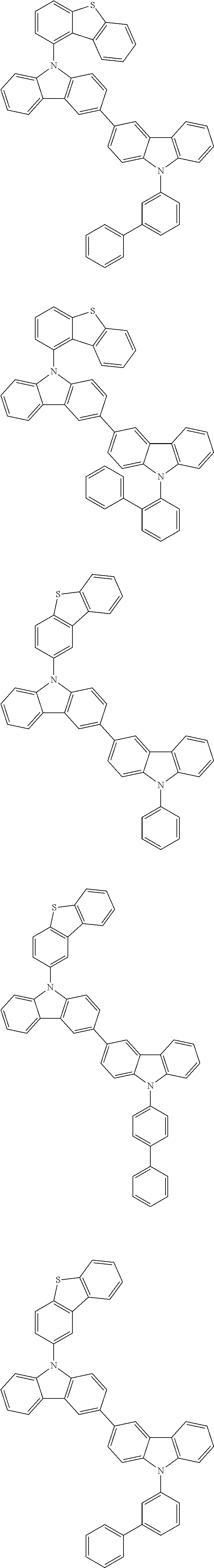 [SMILES: c1ccc(-c2cccc(-n3c4ccccc4c4cc(-c5ccc6c(c5)c5ccccc5n6-c5cccc6sc7ccccc7c56)ccc43)c2)cc1 ; c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

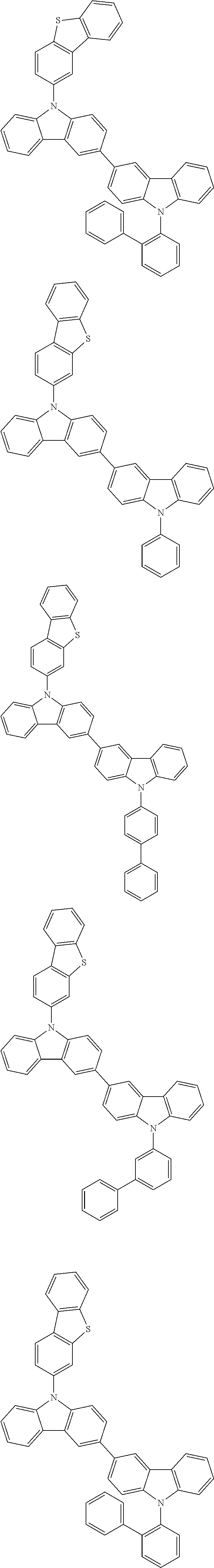 [SMILES: c1ccc(-c2ccccc2-n2c3ccccc3c3cc(-c4ccc5c(c4)c4ccccc4n5-c4ccc5sc6ccccc6c5c4)ccc32)cc1 ; c1ccc(-n2c3ccccc3c3cc(-c4ccc5c(c4)c4ccccc4n5-c4ccc5c(c4)sc4ccccc45)ccc32)cc1 ; c1ccc(-c2ccc(-n3c4ccccc4c4cc(-c5ccc6c(c5)c5ccccc5n6-c5ccc6c(c5)sc5ccccc56)ccc43)cc2)cc1 ; c1ccc(-c2cccc(-n3c4ccccc4c4cc(-c5ccc6c(c5)c5ccccc5n6-c5ccc6c(c5)sc5ccccc56)ccc43)c2)cc1 ; c1ccc(-c2ccccc2-n2c3ccccc3c3cc(-c4ccc5c(c4)c4ccccc4n5-c4ccc5c(c4)sc4ccccc45)ccc32)cc1]

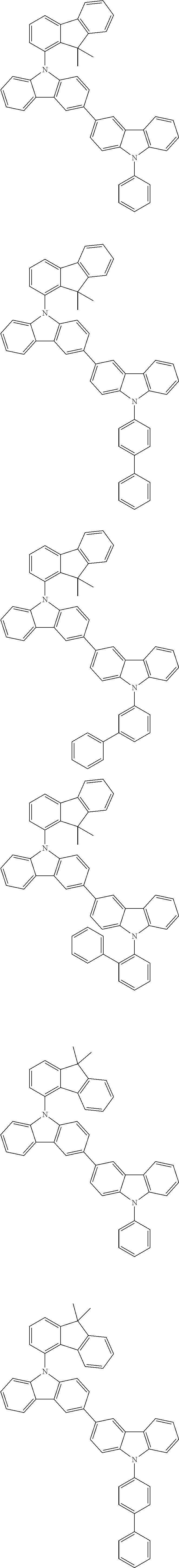

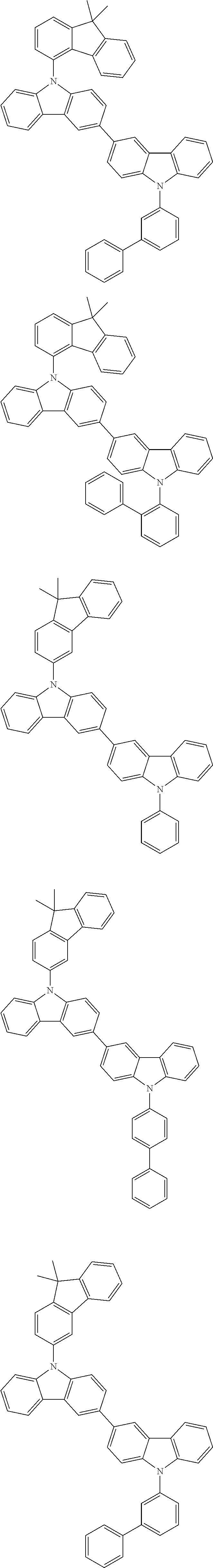

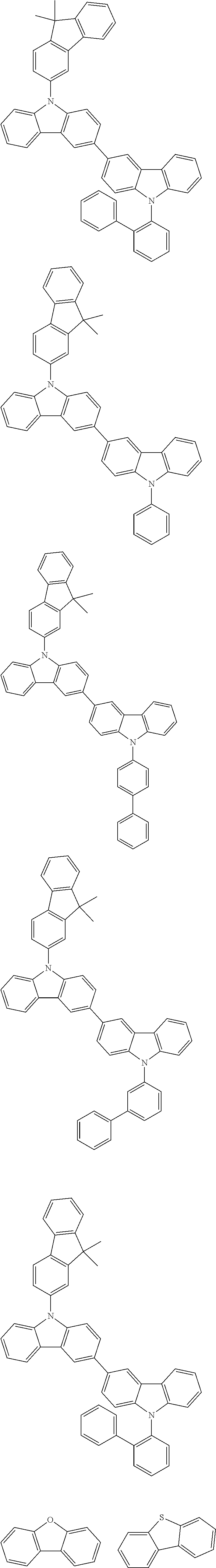

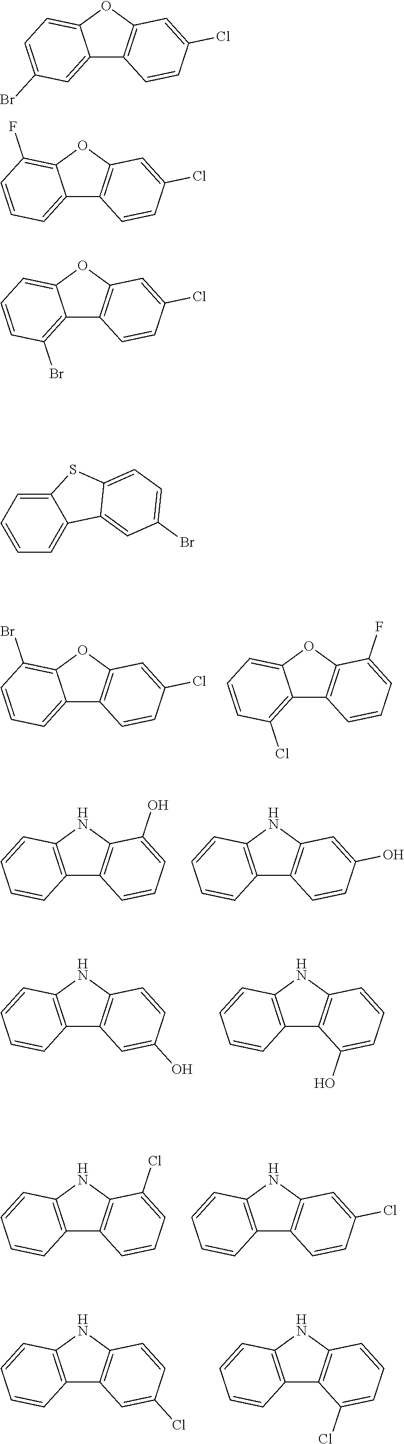

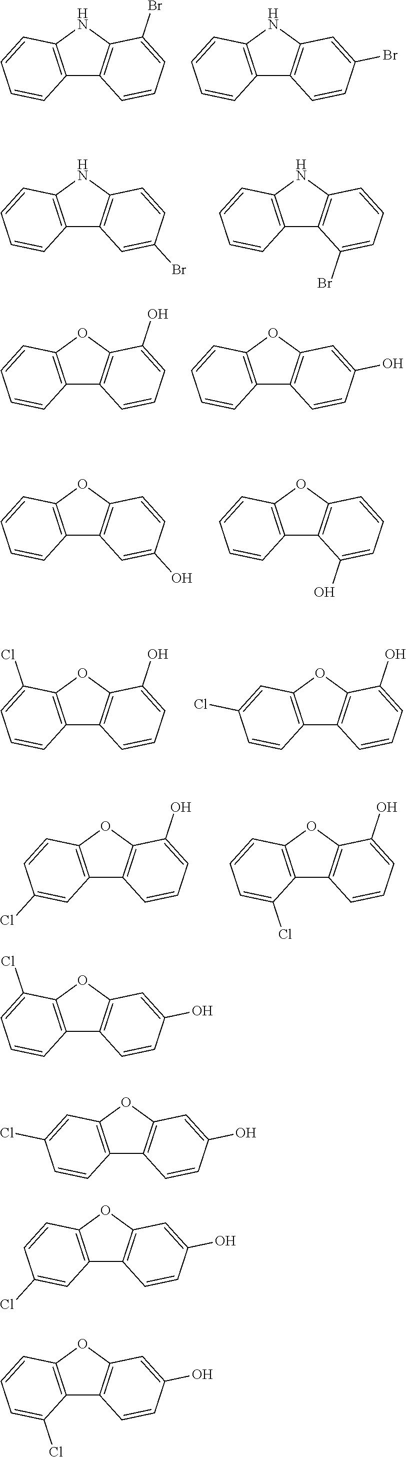

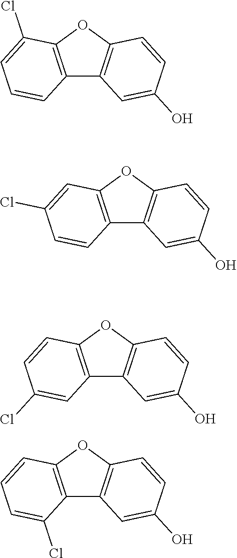

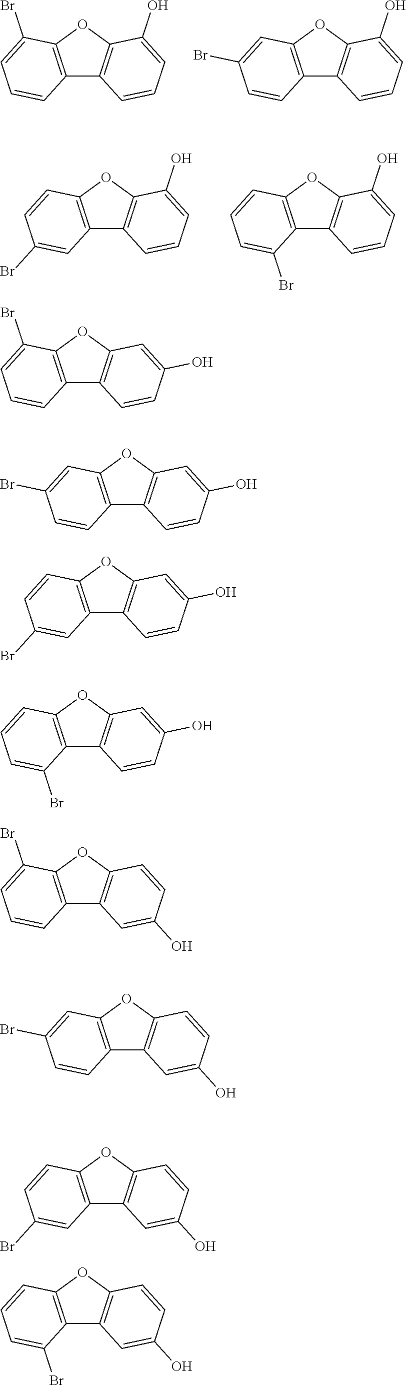

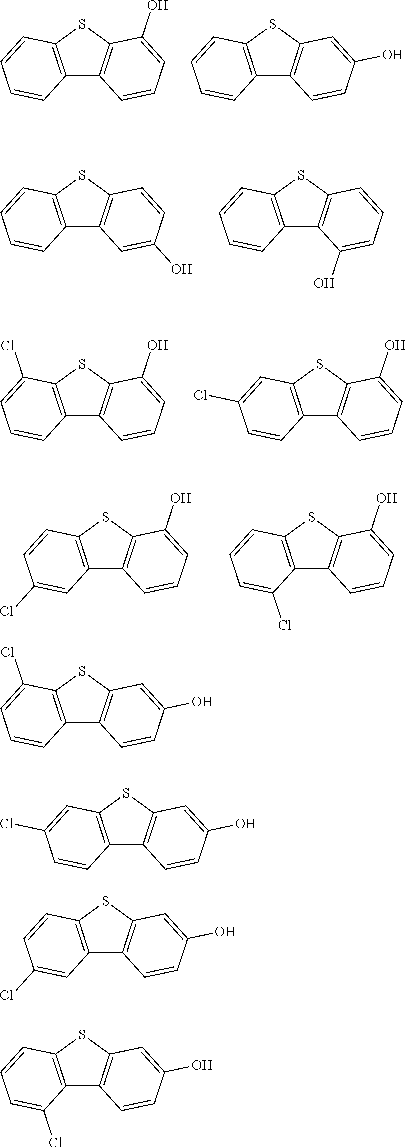

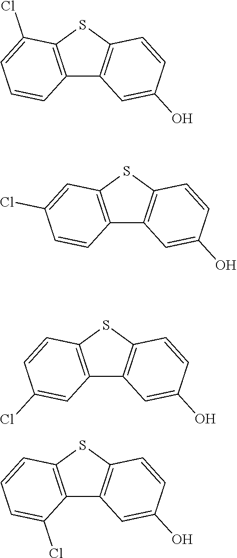

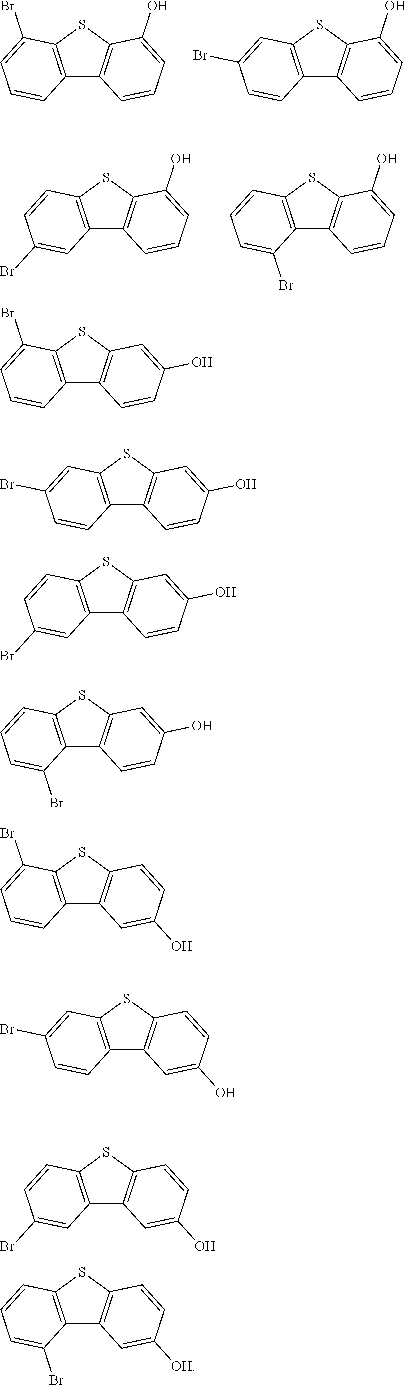

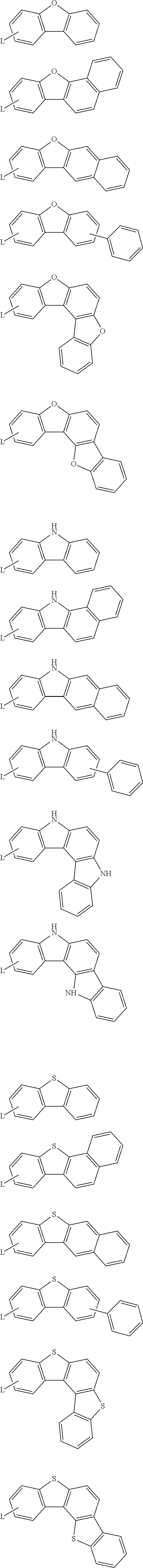

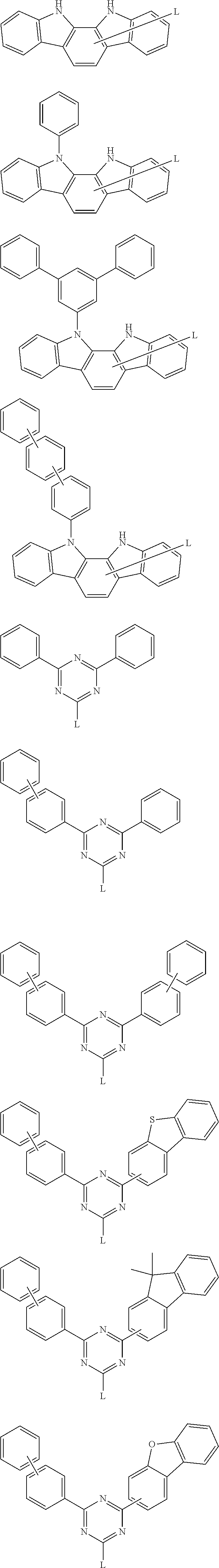

Here, L is a substituent selected from the group consisting of a leaving group, a hydroxyl group, a substituted or unsubstituted amine group, and a cyano group.

The method for producing a deuterated aromatic compound of the present specification may further include substituting the internal air of the reactor with nitrogen or an inert gas.

In the performing of deuteration of the aromatic compound, deuteration may be performed without applying heat at room temperature, or deuteration may be performed by heating the solution. In this case, the room temperature is a natural temperature at which the compound is not heated or cooled, and may be specifically in a range of 20±5° C.

In the method for producing a deuterated aromatic compound of the present specification, the performing of the deuterated reaction of the aromatic compound may include:

preparing a solution including an aromatic compound including one or more aromatic rings, heavy water (D₂O), an organic compound which can be hydrolyzed by the heavy water, and an organic solvent; and

performing the deuterated reaction of the aromatic compound by heating the solution.

The performing of the deuterated reaction of the aromatic compound by heating the reactor may be a step of heating the solution at a temperature of 160° C. or less, 150° C. or less, 140° C. or less, 130° C. or less, 120° C. or less, 110° C. or less, 100° C. or less, 90° C. or less, or 80° C. or more, specifically, a temperature of 80° C. or more and 140° C. or less.

In this case, the deuterium reaction time is reacted for 3 hours or more after the temperature is completely increased. Specifically, the deuterium reaction time may be reacted for 3 hours or more and 24 hours or less, preferably for 6 hours or more and 18 hours or less, after the temperature in the deuterium reaction is completely increased.

The method for producing a deuterated aromatic compound of the present specification further includes obtaining the deuterated aromatic compound after performing the deuteration. The deuteration method may be performed according to a method known in the art, and is not particularly limited.

The higher the deuterium substitution rate of the obtained deuterated aromatic compound, the better the deuterium substitution rate, and specifically, the deuterium substitution rate of the obtained deuterated aromatic compound may be 50% or more, 60% or more, 70% or more, 80% or more, 85% or more, 90% or more, 91% or more, 92% or more, 93% or more, 94% or more, 95% or more, 96% or more, 97% or more, 98% or more, 99% or more or 100%.

The higher the purity of the obtained deuterated aromatic compound, the better the purity, and specifically, the purity of the obtained deuterated aromatic compound may be 90% or more, 91% or more, 92% or more, 93% or more, 94% or more, 95% or more, 96% or more, 97% or more, 98% or more, 99% or more or 100%.

The present specification provides a deuterated aromatic compound produced by the above-described production method.

In an exemplary embodiment of the present specification, the deuterated aromatic compound means an aromatic compound which is substituted with at least one or more deuterium.

In an exemplary embodiment of the present specification, the deuterated aromatic compound includes a substituent selected from the group consisting of a leaving group, a hydroxyl group, a substituted or unsubstituted amine group and a cyano group.

In the present specification, the compound including the leaving group may be an intermediate of a final compound of organic synthesis, and the leaving group means a reaction group which is left based on the final compound, or is chemically modified by being bonded to other reactants. Thus, for the leaving group, the type of leaving group and the position to which the leaving group is bonded are determined by the method of organic synthesis and the position of the substituent of the final compound.

In an exemplary embodiment of the present specification, the leaving group may be selected from the group consisting of a halogen group and a boronic acid group.

In an exemplary embodiment of the present specification, a deuterated aromatic compound including the substituent selected from the group consisting of a leaving group, a hydroxyl group, a substituted or unsubstituted amine group and a cyano group may be any one of the following Chemical Formulae 7 to 10.

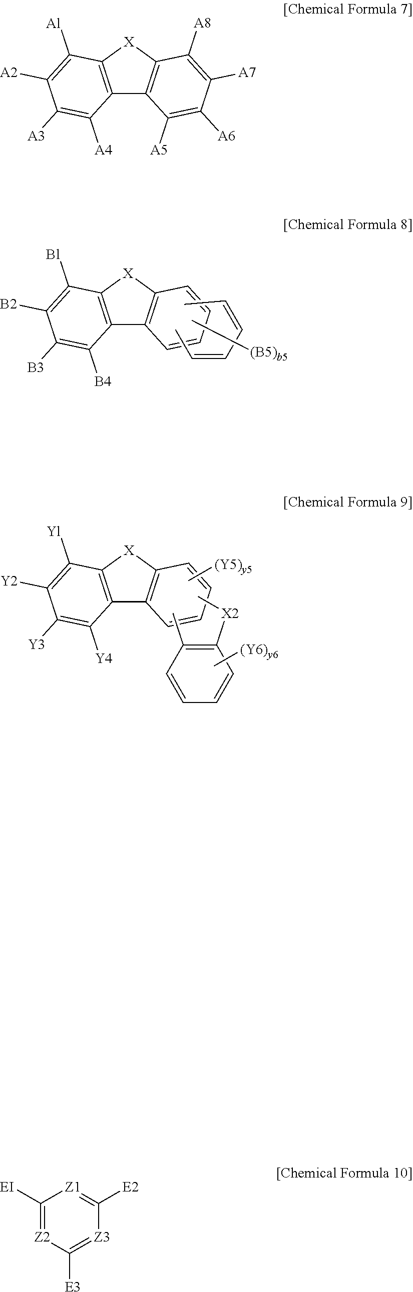

In Chemical Formulae 7 to 10,

X, X1 and X2 are each independently 0, S or NR, wherein R is hydrogen; deuterium; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

at least one of A1 to A8 is deuterium, at least one is a substituent selected from the group consisting of a leaving group, a hydroxyl group, a substituted or unsubstituted amine group and a cyano group, and the others are each independently hydrogen; a leaving group; a hydroxyl group; a substituted or unsubstituted amine group; a substituted or unsubstituted alkyl group; a substituted or unsubstituted cycloalkyl group; a cyano group; a substituted or unsubstituted aryl group; or a substituted or unsubstituted heterocyclic group,

at least one of B1 to B5 is deuterium, at least one is a substituent selected from the group consisting of a leaving group, a hydroxyl group, a substituted or unsubstituted amine group and a cyano group, and the others are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

at least one of E1 to E3 is deuterium, at least one is a substituent selected from the group consisting of a leaving group, a hydroxyl group, a substituted or unsubstituted amine group and a cyano group, and the others are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

at least one of Y1 to Y6 is deuterium, at least one is a substituent selected from the group consisting of a leaving group, a hydroxyl group, a substituted or unsubstituted amine group and a cyano group, and the others are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,

at least one of Z1 to Z3 is N, and the others are each independently CH or N,

b5 is an integer from 1 to 6, and when b5 is 2 or higher, B5's are the same as or different from each other,

y5 is 1 or 2, and when y5 is 2, Y5's are the same as or different from each other, and

y6 is an integer from 1 to 4, and when y6 is 2 or higher, Y6's are the same as or different from each other.

In an exemplary embodiment of the present specification, the compounds of Chemical Formulae 7 to 10 each have a substituent selected from the group consisting of a leaving group, a hydroxyl group, a substituted or unsubstituted amine group and a cyano group.

In an exemplary embodiment of the present specification, a deuterated aromatic compound including the substituent selected from the group consisting of a leaving group, a hydroxyl group, a substituted or unsubstituted amine group and a cyano group is any one of the following structures, and the structures are each substituted with one or more deuteriums.

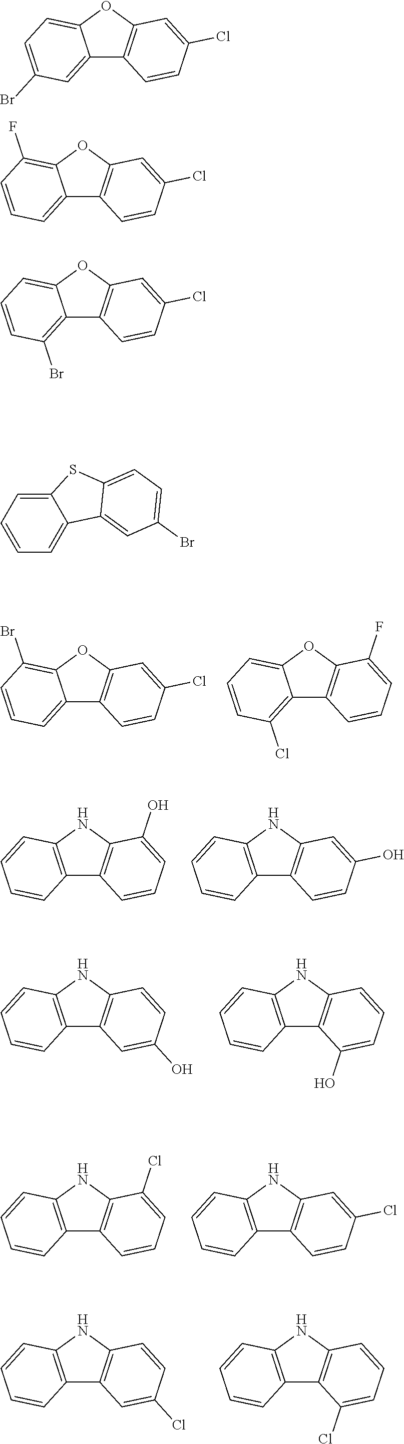

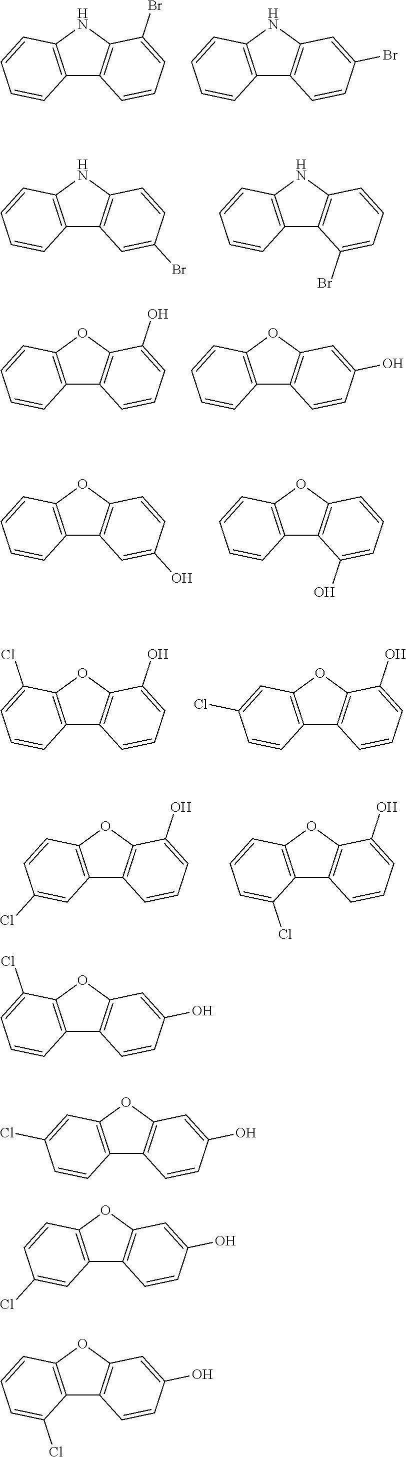

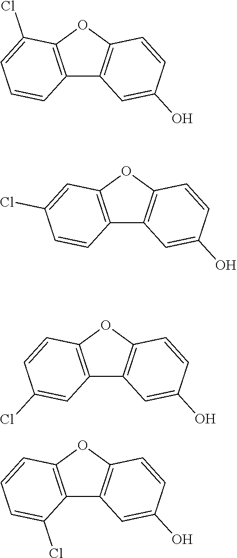

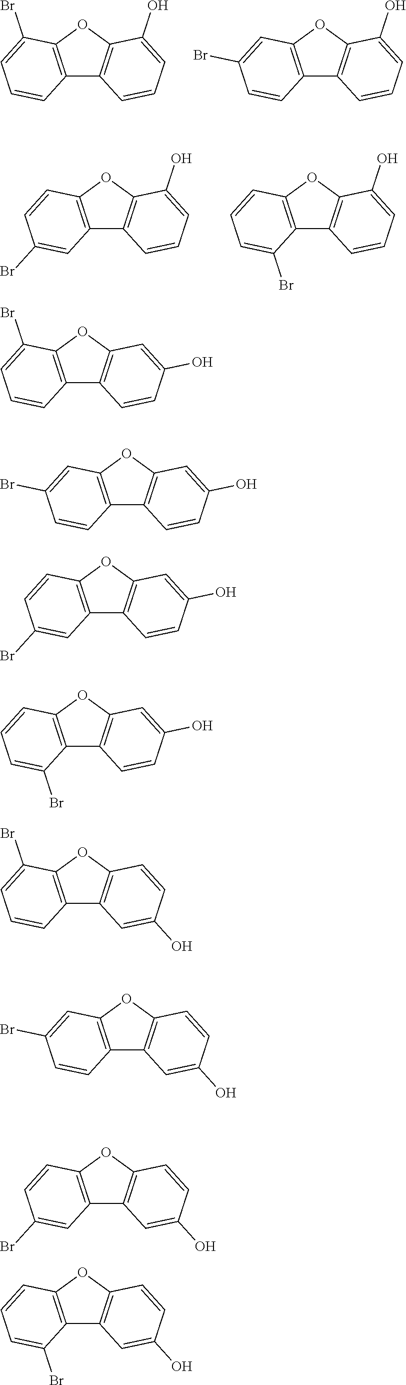

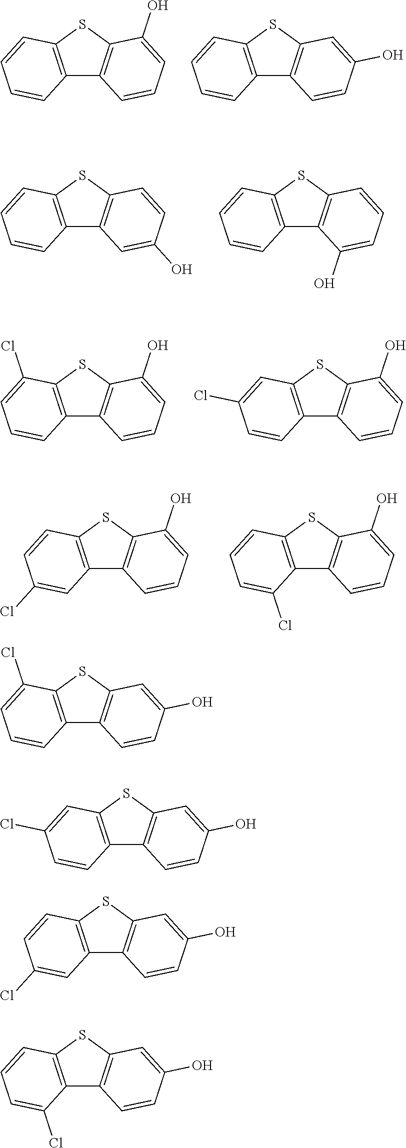

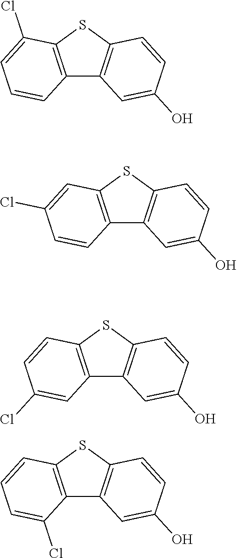

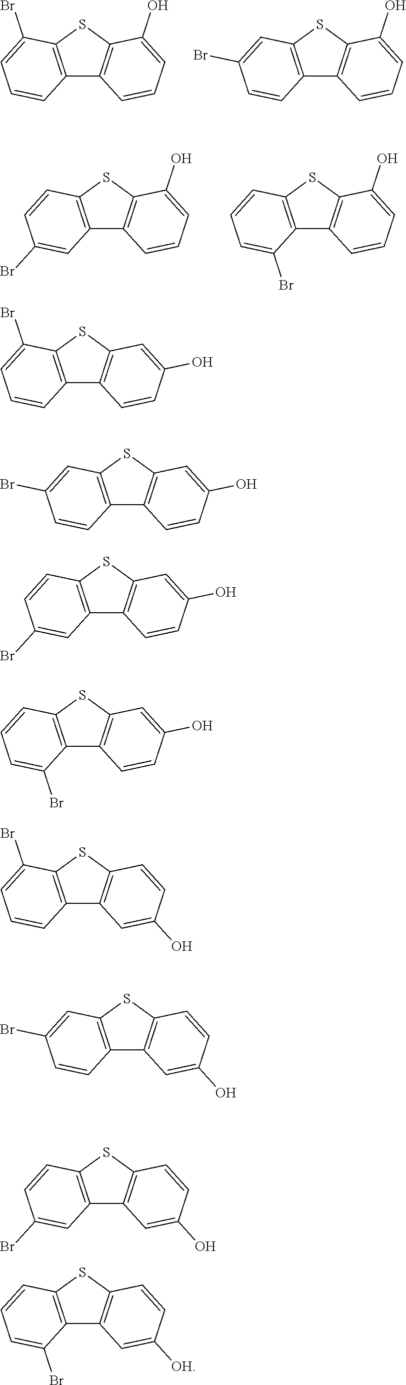

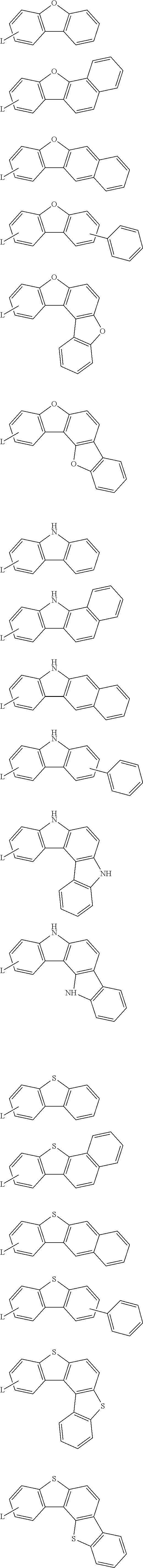

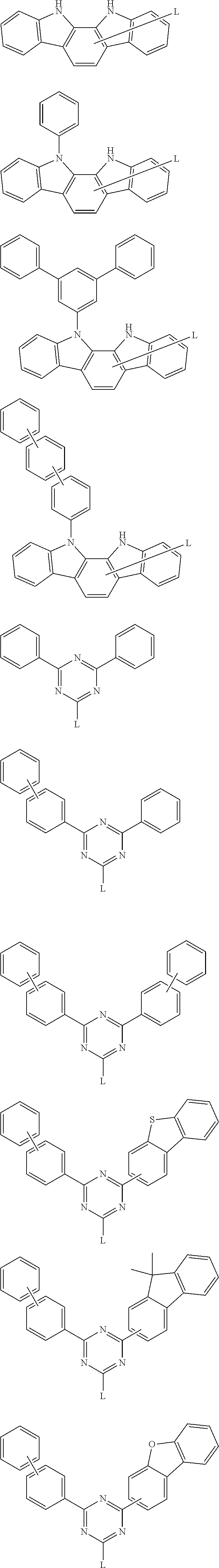

Here, L is a substituent selected from the group consisting of a leaving group, a hydroxyl group, a substituted or unsubstituted amine group, and a cyano group.

Theoretically, when all the hydrogens in the deuterated compound is substituted with deuterium, that is, when the deuterium substitution rate is 100%, the service life characteristics are most ideally improved. However, there are problems such as the need for extreme conditions due to steric hindrance and the destruction of the compound before the compound is deuterated due to side reactions, and in reality, it is difficult to obtain all the hydrogen of a compound at a deuterated substitution rate of 100%. Even when a deuterated substitution rate of nearly 100% is obtained, the efficiency compared to investment is not good in consideration of process time, cost, and the like.

In the present specification, since a deuterated compound produced by a deuterated reaction and having one or more deuteriums is produced as a composition having two or more isotopes having different molecular weights depending on the number of substituted deuteriums, the position where deuterium is substituted in the structure will be omitted.

In the compound having the structure, at least one of the positions which are indicated by hydrogen or in which substituted hydrogen is omitted may be substituted with deuterium.

The present specification provides a deuterated reaction composition including an aromatic compound including one or more aromatic rings, heavy water (D₂O), an organic compound which can be hydrolyzed by the heavy water, and an organic solvent.

For the deuterated reaction composition, the description on the solution in above-described production method may be cited.

In an exemplary embodiment of the present specification, the organic compound which can be hydrolyzed by the heavy water may include at least one compound of the following Chemical Formulae 1 to 4.

R1-C(O)OC(O)—R2 [Chemical Formula 1]

R3-S(O₂)OS(O₂)—R4 [Chemical Formula 2]

R5-C(O)O—R6 [Chemical Formula 3]

R7-CONH—R8 [Chemical Formula 4]

In Chemical Formulae 1 to 4,

R1 to R8 are the same as or different from each other, and are each independently a monovalent organic group.

In an exemplary embodiment of the present specification, R1 to R8 are the same as or different from each other, and may be each independently an alkyl group which is unsubstituted or substituted with a halogen group; or an aryl group which is unsubstituted or substituted with a halogen group.

In an exemplary embodiment of the present specification, R1 to R8 are the same as or different from each other, and may be each independently a substituent of the following Chemical Formula 5 or 6.

—(CH₂)ₗ(CF₂)ₙ(CF₃)ₙ(CH₃)₁₋ₙ [Chemical Formula 5]

—C(H)ₐ((CH₂)ₗ(CF₂)ₙCF₃)₃₋ₐ [Chemical Formula 6]

In Chemical Formulae 5 and 6,

l and m are each an integer from 0 to 10, and

n and a are each 0 or 1.

In an exemplary embodiment of the present specification, the organic compound which can be hydrolyzed by the heavy water may include at least one of trifluoromethanesulfonic anhydride, trifluoroacetic anhydride, acetic anhydride and methanesulfonic anhydride.

According to an exemplary embodiment of the present specification, the organic solvent may be selected from the group consisting of a hydrocarbon chain which is unsubstituted or substituted with a halogen group; an aliphatic hydrocarbon ring which is unsubstituted or substituted with an alkyl group; an aromatic hydrocarbon ring which is unsubstituted or substituted with an alkyl group; a straight-chained or branched heterochain; a substituted or unsubstituted aliphatic hetero ring; and a substituted or unsubstituted aromatic hetero ring. Specifically, the organic solvent includes at least one of an oxygen element and a sulfur atom, and is selected from the group consisting of a substituted or unsubstituted hetero ring; a substituted or unsubstituted alkyl acetate; alkyl ketone; alkylsulfoxide; a lactone having 4 to 10 carbon atoms; alkylamide; a glycol having 4 to 10 carbon atoms; dioxane; an acetic acid which is unsubstituted or substituted with alkoxy.

The organic solvent may be selected from the group consisting of ethyl acetate, acetone, cyclohexanone, methyl ethyl ketone, tetrahydrofuran, tetrahydropyran, cyclopentanone, 1,2-dioxane, 1,3-dioxane, 1,4-dioxane, N,N-dimethylformamide (DMF), dimethyl sulfoxide (DMSO), 1,2-dimethoxyethane, diglyme, γ-butyrolactone, γ-valerolactone, methyl ethyl diglycol (MEDG), propylene glycol methyl ether (PGME), propylene glycol methyl ether acetate (PGMEA), ethyl lactate, cyclohexane, methylcyclohexane, ethylcyclohexane, diethyl ether, 1,2-dimethoxyethane, decalin, hexane, heptane, toluene, xylene, 1,3,5-trimethylbenzene, dichloromethane, 1,2-dichloroethane, 1,1,2,2-tetrachloroethane, tetrachloroethylene and 2-methoxyacetic acid.

The present specification provides an electronic device including the above-described deuterated aromatic compound.

The present specification provides a method for manufacturing an electronic device, the method including: manufacturing an electronic device using the above-described deuterated aromatic compound.

For the electronic device and the method for manufacturing an electronic device, the description on the composition may be cited, and the repeated description will be omitted.

The electronic device is not particularly limited as long as the electronic device can use the above-described deuterated aromatic compound, and may be, for example, an organic light emitting device, an organic phosphorescent device, an organic solar cell, an organic photo conductor, an organic transistor, or the like.

The electronic device includes: a first electrode; a second electrode provided to face the first electrode; and an organic material layer having one or more layers provided between the first electrode and the second electrode, and one or more layers of the organic material layer may include the above-described deuterated aromatic compound.

The present specification provides an organic light emitting device including the above-described deuterated aromatic compound.

In an exemplary embodiment of the present specification, the organic light emitting device includes: a first electrode; a second electrode provided to face the first electrode; and an organic material layer provided between the first electrode and the second electrode, in which the organic material layer includes the deuterated aromatic compound.

In an exemplary embodiment of the present specification, the organic material layer includes a light emitting layer including the deuterated aromatic compound.

The organic material layer of the organic light emitting device of the present specification may also be composed of a single-layered structure, but may be composed of a multi-layered structure in which two or more organic material layers are stacked. For example, the organic material layer of the present specification may be composed of one to three layers. Further, the organic light emitting device of the present specification may have a structure including a hole injection layer, a hole transport layer, a light emitting layer, an electron transport layer, an electron injection layer, and the like as organic material layers. However, the structure of the organic light emitting device is not limited thereto, and may include a fewer number of organic layers.

When the organic light emitting device includes a plurality of organic material layers, the organic material layers may be formed of the same material or different materials.

For example, the organic light emitting device of the present specification may be manufactured by sequentially stacking a positive electrode, an organic material layer, and a negative electrode on a substrate. In this case, the organic light emitting device may be manufactured by depositing a metal or a metal oxide having conductivity, or an alloy thereof on a substrate to form a positive electrode, forming an organic material layer including a hole injection layer, a hole transport layer, a light emitting layer, and an electron transport layer thereon, and then depositing a material, which may be used as a negative electrode, thereon, by using a physical vapor deposition (PVD) method such as sputtering or e-beam evaporation. In addition to the method described above, an organic light emitting device may be made by sequentially depositing a negative electrode material, an organic material layer, and a positive electrode material on a substrate.

Further, the compound of Chemical Formula 1 may be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

In an exemplary embodiment of the present specification, the first electrode is a positive electrode, and the second electrode is a negative electrode.

According to another exemplary embodiment, the first electrode is a negative electrode, and the second electrode is a positive electrode.

In another exemplary embodiment, the organic light emitting device may be a normal type organic light emitting device in which a positive electrode, an organic material layer having one or more layers, and a negative electrode are sequentially stacked on a substrate.

In still another exemplary embodiment, the organic light emitting device may be an inverted type organic light emitting device in which a negative electrode, an organic material layer having one or more layers, and a positive electrode are sequentially stacked on a substrate.

In the present specification, the materials for the negative electrode, the organic material layer and the positive electrode are not particularly limited except for including an aromatic compound deuterated in at least one layer of the organic material layer, and a material known in the art may be used.

In the present specification, the above-described deuterated aromatic compound may be used by a principle which is similar to the principle applied to an organic light emitting device, even in an electronic device including an organic phosphorescent device, an organic solar cell, an organic photo conductor, an organic transistor, and the like. For example, the organic solar cell may have a structure including a negative electrode, a positive electrode, and a photoactive layer provided between the negative electrode and the positive electrode, and the photoactive layer may include the selected deuterated compound.

### EXAMPLES

Hereinafter, the present specification will be described in more detail through Examples. However, the following Examples are provided only for exemplifying the present specification, but are not intended to limit the present specification.

### EXAMPLES

### Example 1

5 g of 11,12-dihydroindolo [2,3-a]carbazole, 30 ml of heavy water (D₂O), 10 g of methanesulfonic anhydride and 50 ml of dimethyl sulfoxide was put into a flask and stirred at room temperature for 1 hour, and then allowed to react at 60° C. to 100° C. for 18 hours. After completion of the reaction, the temperature was lowered to 5° C. or less, and then the resulting product was neutralized by adding potassium carbonate thereto to adjust the pH to 7 to 8. When the resulting product became neutralized, the reactant was precipitated as a solid as the solubility was reduced. The precipitate was filtered and dissolved in tetrahydrofuran. After the residual moisture was removed over magnesium sulfate (MgSO₄), the residue was filtered and 11,12-dihydroindolo[2,3-a]carbazole substituted with deuterium was obtained by removing the solvent using a rotary evaporator.

### Example 2

11,12-dihydroindolo[2,3-a]carbazole substituted with deuterium was obtained by changing the organic solvent to tetrahydrofuran instead of dimethyl sulfoxide, using the same method as in Example 1.

### Example 3

11,12-dihydroindolo[2,3-a]carbazole substituted with deuterium was obtained by changing the organic solvent to 1,4-dioxane instead of dimethyl sulfoxide, using the same method as in Example 1.

### Example 4

11,12-dihydroindolo[2,3-a]carbazole substituted with deuterium was obtained by changing the organic solvent to methylcyclohexane instead of dimethyl sulfoxide using the same method as in Example 1.

### Example 5

11,12-dihydroindolo[2,3-a]carbazole substituted with deuterium was obtained by changing the organic solvent to 1,2-dichloroethane instead of dimethyl sulfoxide, using the same method as in Example 1.

### Example 6

11,12-dihydroindolo[2,3-a]carbazole substituted with deuterium was obtained by changing the organic solvent to xylene instead of dimethyl sulfoxide, using the same method as in Example 1.

### Example 7

11,12-dihydroindolo[2,3-a]carbazole substituted with deuterium was obtained by changing methnaesulfonic anhydride to trifluoromethanesulfonic anhydride, using the same method as in Example 1.

### Example 8

11,12-dihydroindolo[2,3-a]carbazole substituted with deuterium was obtained by changing methnaesulfonic anhydride and dimethyl sulfoxide to trifluoroacetic anhydride and xylene, respectively, using the same method as in Example 1.

### Example 9

5 g of carbazole, 32 ml of heavy water (D₂O), 8 g of methanesulfonic anhydride and 50 ml of dimethyl sulfoxide was put into a flask and stirred at room temperature for 1 hour, and then allowed to react at 60° C. to 100° C. for 18 hours. After completion of the reaction, the temperature was lowered to 5° C. or less, and then the resulting product was neutralized by adding potassium carbonate thereto to adjust the pH to 7 to 8. When the resulting product became neutralized, the reactant was precipitated as a solid while the solubility was reduced. The precipitate was filtered and dissolved in tetrahydrofuran. After the residual moisture was removed over magnesium sulfate (MgSO₄), the residue was filtered and carbazole substituted with deuterium was obtained by removing the solvent using a rotary evaporator.

### Example 10

Carbazole substituted with deuterium was obtained by changing the organic solvent to tetrahydrofuran instead of dimethyl sulfoxide, using the same method as in Example 9.

### Example 11

Carbazole substituted with deuterium was obtained by changing the organic solvent to 1,4-dioxane instead of dimethyl sulfoxide, using the same method as in Example 9.

### Example 12

Carbazole substituted with deuterium was obtained by changing the organic solvent to methylcyclohexane instead of dimethyl sulfoxide, using the same method as in Example 9.

### Example 13

Carbazole substituted with deuterium was obtained by changing the organic solvent to 1,2-dichloroethane instead of dimethyl sulfoxide, using the same method as in Example 9.

### Example 14

Carbazole substituted with deuterium was obtained by changing the organic solvent to xylene instead of dimethyl sulfoxide, using the same method as in Example 9.

### Example 15

Carbazole substituted with deuterium was obtained by changing methanesulfonic anhydride to trifluoromethanesulfonic anhydride, using the same method as in Example 9.

### Example 16

Carbazole substituted with deuterium was obtained by changing methanesulfonic anhydride and dimethyl sulfoxide to trifluoroacetic anhydride and xylene, respectively, using the same method as in Example 9.

### Example 17

5 g of 2-bromodibenzofuran, 16 ml of heavy water (D₂O), 10.5 g of methanesulfonic anhydride and 40 ml of dimethyl sulfoxide was put into a flask and stirred at room temperature for 1 hour, and then allowed to react at 80° C. to 100° C. for 18 hours. After completion of the reaction, the temperature was lowered to 5° C. or less, and then the resulting product was neutralized by adding potassium carbonate thereto to adjust the pH to 7 to 8. When the resulting product became neutralized, the reactant was precipitated as a solid while the solubility was reduced. The precipitate was filtered and dissolved in ethyl acetate. After the residual moisture was removed over magnesium sulfate (MgSO₄), the residue was filtered and 2-bromodibenzofuran substituted with deuterium was obtained by removing the solvent using a rotary evaporator.

### Example 18

2-bromodibenzofuran substituted with deuterium was obtained by changing the organic solvent to tetrahydrofuran instead of dimethyl sulfoxide, using the same method as in Example 17.

### Example 19

2-bromodibenzofuran substituted with deuterium was obtained by changing the organic solvent into 1,4-dioxane instead of dimethyl sulfoxide using the same method as in Example 17.

### Example 20

2-bromodibenzofuran substituted with deuterium was obtained by changing the organic solvent to methylcyclohexane instead of dimethyl sulfoxide using the same method as in Example 17.

### Example 21

2-bromodibenzofuran substituted with deuterium was obtained by changing the organic solvent to 1,2-dichloroethane instead of dimethyl sulfoxide, using the same method as in Example 17.

### Example 22

2-bromodibenzofuran substituted with deuterium was obtained by changing the organic solvent to xylene instead of dimethyl sulfoxide, using the same method as in Example 17.

### Example 23

2-bromodibenzofuran substituted with deuterium was obtained by changing methanesulfonic anhydride to trifluoromethanesulfonic anhydride, using the same method as in Example 17.

### Example 24

2-bromodibenzofuran substituted with deuterium was obtained by changing methanesulfonic anhydride and dimethyl sulfoxide to trifluoroacetic anhydride and xylene, respectively, using the same method as in Example 17.

### Example 25

5 g of 2-chloro-4,6-diphenyl-1,3,5-triazine, 20.5 ml of heavy water (D₂O), 13 g of methanesulfonic anhydride and 40 ml of dimethyl sulfoxide was put into a flask and stirred at room temperature for 1 hour, and then allowed to react at 80° C. to 100° C. for 18 hours. After completion of the reaction, the temperature was lowered to 5° C. or less, and then the resulting product was neutralized by adding potassium carbonate thereto to adjust the pH to 7 to 8. When the resulting product became neutralized, the reactant was precipitated as a solid while the solubility was reduced. The precipitate was filtered and dissolved in ethyl acetate. After the residual moisture was removed over magnesium sulfate (MgSO₄), the residue was filtered and 2-chloro-4,6-diphenyl-1,3,5-triazine substituted with deuterium was obtained by removing the solvent using a rotary evaporator.

### Example 26

2-chloro-4,6-diphenyl-1,3,5-triazine substituted with deuterium was obtained by changing the organic solvent to tetrahydrofuran instead of dimethyl sulfoxide, using the same method as in Example 25.

### Example 27

2-chloro-4,6-diphenyl-1,3,5-triazine substituted with deuterium was obtained by changing the organic solvent to 1,4-dioxane instead of dimethyl sulfoxide, using the same method as in Example 25.

### Example 28

2-chloro-4,6-diphenyl-1,3,5-triazine substituted with deuterium was obtained by changing the organic solvent to methylcyclohexane instead of dimethyl sulfoxide, using the same method as in Example 25.

### Example 29

2-chloro-4,6-diphenyl-1,3,5-triazine substituted with deuterium was obtained by changing the organic solvent to 1,2-dichloroethane instead of dimethyl sulfoxide, using the same method as in Example 25.

### Example 30

2-chloro-4,6-diphenyl-1,3,5-triazine substituted with deuterium was obtained by changing the organic solvent into xylene instead of dimethyl sulfoxide using the same method as in Example 25.

### Example 31

2-chloro-4,6-diphenyl-1,3,5-triazine substituted with deuterium was obtained by changing methanesulfonic anhydride to trifluoromethanesulfonic anhydride, using the same method as in Example 25.

### Example 32

2-chloro-4,6-diphenyl-1,3,5-triazine substituted with deuterium was obtained by changing methanesulfonic anhydride and dimethyl sulfoxide to trifluoroacetic anhydride and xylene, respectively, using the same method as in Example 25.

### Comparative Example 1

2 g of 11,12-dihydroindolo [2,3-a]carbazole, 30 ml of heavy water (D₂O), 0.5 g of 10% Pd/C, and 10 ml of a solvent in which toluene and xylene were mixed at a ratio of 6:4 were put into a high-pressure reactor and the inside of the reactor was sealed by covering the head of the reactor. A gas including 4% hydrogen was blown into the reactant for 3 to 5 minutes per minute with stirring. And then, the atmosphere in the reactor was maintained with a gas atmosphere including 4% hydrogen, and the reaction was performed at an oil bath temperature of 145° C. for 24 hours. After the deuterium substitution reaction was completed, the temperature was lowered, the inside of the reactor was substituted with outside air, and then the temperature of the oil bath was increased to 160° C., and the dehydrogenation reaction was performed for 17 hours. After the dehydrogenation reaction was completed, the temperature was lowered, filtration was performed to remove the catalyst, and then heavy water was removed using MgSO₄, and then 11,12-dihydroindolo [2,3-a]carbazole substituted with deuterium was obtained by removing the solvent using a rotary evaporator.

### Comparative Example 2

2 g of 11,12-dihydroindolo [2,3-a]carbazole, 30 ml of heavy water (D₂O), 0.5 g of 10% Pd/C, and 10 ml of a solvent in which toluene and xylene were mixed at a ratio of 6:4 were put into a high-pressure reactor and the inside of the reactor was sealed by covering the head of the reactor. 100% hydrogen gas was blown into the reactant for 3 to 5 minutes per minute with stirring. And then, the atmosphere in the reactor was maintained with the atmosphere of a gas including 4% hydrogen, and the reaction was performed at an oil bath temperature of 160° C. for 24 hours. After the deuterium substitution reaction was completed, the temperature was lowered, the inside of the reactor was substituted with outside air, and then the temperature of the oil bath was increased to 160° C., and the dehydrogenation reaction was performed for 17 hours. After the dehydrogenation reaction was completed, the temperature was lowered, filtration was performed to remove the catalyst, and then heavy water was removed using MgSO₄, and then 11,12-dihydroindolo [2,3-a]carbazole substituted with deuterium was obtained by removing the solvent using a rotary evaporator.

### Comparative Example 3

A deuterium substitution reaction was performed by adding 2-bromo-dibenzofuran instead of 11,12-dihydroindolo [2,3-a]carbazole using the same methods as in Comparative Example 1. As a result, 2-bromodibenzofuran substituted with deuterium was obtained, but dibenzofuran substituted with deuterium, which lost most of the bromine group could be confirmed.

### Experimental Example 1

The purity, deuterium substitution rate, and hydrogenated compound proportion for Examples 1 to 32 and Comparative Examples 1 to 3 were measured, and the results are shown in the following Table 1.

The purity and hydrogenated compound proportion were obtained by dissolving the completely reacted specimen in a tetrahydrofuran solvent for HPLC to integrate the spectrum at a wavelength of 254 nm through HPLC. In this case, as a mobile phase solvent, a solvent in which acetonitrile and tetrahydrofuran were mixed at a ratio of 5:5 and 1% formic acid was mixed and water were used.

A sample specimen obtained by quantifying a specimen completely subjected to deuterated reaction and dissolving the specimen in a solvent for NMR measurement, and an internal standard specimen obtained by quantifying any compound whose peak does not overlap with the compound before the deuterated reaction in the same amount as the above specimen and dissolving the compound in the same solvent for NMR measurement were prepared. NMR measurement graphs were obtained each using ¹H-NMR for the prepared sample specimen and internal standard specimen.

When the ¹H-NMR peak was assigned, a relative integration value for each position of the specimen completely subjected to deuterated reaction was obtained by setting the internal standard peak to 1.

When the specimen completely subjected to deuterated reaction is substituted with deuterium at all positions, no peak related to hydrogen appears, and in this case, the deuterium substitution rate is determined to be 100%. In contrast, when hydrogen at all positions is not substituted with deuterium, a peak of hydrogen that has not been substituted with deuterium will appear.

Based on this result, in the present experiment, a deuterium substitution rate is obtained by subtracting an integration value of a peak due to unsubstituted hydrogen in the NMR measurement graph of the sample specimen from an integration value of a peak related to hydrogen in the NMR measurement graph of the internal standard specimen in which deuterium is not substituted. This value is an integration value relative to each position, does not appear as the corresponding peak due to substitution with deuterium, and indicates a ratio of substitution with deuterium.

And then, a substitution rate for each position of the specimen was calculated using the weight of the specimen used when the ¹H-NMR measurement sample is prepared, the weight of the internal standard, and the relative integration value.

TABLE 1

| | | Organic | Hydrolyzed |
|---|---|---|---|
| | Reactant | solvent | compound |
| Example 1 | 11,12-dihydroindolo | Dimethylsulfoxide | Methanesulfonic anhydride |
| | [2,3-a]carbazole | | |
| Example 2 | 11,12-dihydroindolo | Tetrahydrofuran | Methanesulfonic anhydride |
| | [2,3-a]carbazole | | |
| Example 3 | 11,12-dihydroindolo | 1,4-dioxane | Methanesulfonic anhydride |
| | [2,3-a]carbazole | | |
| Example 4 | 11,12-dihydroindolo | Methylcyclohexane | Methanesulfonic anhydride |
| | [2,3-a]carbazole | | |
| Example 5 | 11,12-dihydroindolo | 1,2-dichloroethane | Methanesulfonic anhydride |
| | [2,3-a]carbazole | | |
| Example 6 | 11,12-dihydroindolo | Xylene | Methanesulfonic anhydride |
| | [2,3-a]carbazole | | |
| Example 7 | 11,12-dihydroindolo | Dimethylsulfoxide | Trifluoromethanesulfonic anhydride |
| | [2,3-a]carbazole | | |
| Example 8 | 11,12-dihydroindolo | Xylene | Trifluoroacetic anhydride |
| | [2,3-a]carbazole | | |
| Example 9 | Carbazole | Dimethylsulfoxide | Methanesulfonic anhydride |
| Example 10 | Carbazole | Tetrahydrofuran | Methanesulfonic anhydride |
| Example 11 | Carbazole | 1,4-dioxane | Methanesulfonic anhydride |
| Example 12 | Carbazole | Methylcyclohexane | Methanesulfonic anhydride |
| Example 13 | Carbazole | 1,2-dichloroethane | Methanesulfonic anhydride |
| Example 14 | Carbazole | Xylene | Methanesulfonic anhydride |
| Example 15 | Carbazole | Dimethylsulfoxide | Trifluoromethanesulfonic anhydride |
| Example 16 | Carbazole | Xylene | Trifluoroacetic anhydride |
| Example 17 | 2-bromo-dibenzofuran | Dimethylsulfoxide | Methanesulfonic anhydride |
| Example 18 | 2-bromo-dibenzofuran | Tetrahydrofuran | Methanesulfonic anhydride |
| Example 19 | 2-bromo-dibenzofuran | 1,4-dioxane | Methanesulfonic anhydride |
| Example 20 | 2-bromo-dibenzofuran | Methylcyclohexane | Methanesulfonic anhydride |
| Example 21 | 2-bromo-dibenzofuran | 1,2-dichloroethane | Methanesulfonic anhydride |
| Example 22 | 2-bromo-dibenzofuran | Xylene | Methanesulfonic anhydride |
| Example 23 | 2-bromo-dibenzofuran | Dimethylsulfoxide | Trifluoromethanesulfonic anhydride |
| Example 24 | 2-bromo-dibenzofuran | Xylene | Trifluoroacetic anhydride |
| Example 25 | 2-chloro-4,6- | Dimethylsulfoxide | Methanesulfonic anhydride |
| | diphenyl-1,3,5-triazine | | |
| Example 26 | 2-chloro-4,6- | Tetrahydrofuran | Methanesulfonic anhydride |
| | diphenyl-1,3,5-triazine | | |
| Example 27 | 2-chloro-4,6- | 1,4-dioxane | Methanesulfonic anhydride |
| | diphenyl-1,3,5-triazine | | |
| Example 28 | 2-chloro-4,6- | Methylcyclohexane | Methanesulfonic anhydride |
| | diphenyl-1,3,5-triazine | | |
| Example 29 | 2-chloro-4,6- | 1,2-dichloroethane | Methanesulfonic anhydride |
| | diphenyl- | | |
| | 1,3,5-triazine | | |
| Example 30 | 2-chloro-4,6- | Xylene | Methanesulfonic anhydride |
| | diphenyl- | | |
| | 1,3,5-triazine | | |
| Example 31 | 2-chloro-4,6-diphenyl- | Dimethylsulfoxide | Trifluoromethanesulfonic anhydride |
| | 1,3,5-triazine | | |
| Example 32 | 2-chloro-4,6-diphenyl- | Xylene | Trifluoroacetic anhydride |
| | 1,3,5-triazine | | |
| Comparative | 11,12-dihydroindolo | Toluene, | — |
| Example 1 | [2,3-a]carbazole | xylene (6:4) | |
| Comparative | 11,12-dihydroindolo | Toluene, | — |
| Example 2 | [2,3-a]carbazole | xylene (6:4) | |
| Comparative | 2-bromo-dibenzofuran | Toluene, | — |
| Example 3 | | xylene (6:4) | |

| | | | Hydrogenated | | |
|---|---|---|---|---|---|
| | | Deuterium | compound | Reaction | Reaction |
| | Purity | substitution | proportion | temperature | pressure |
| | (%) | rate (%) | (%) | (° C.) | (bar) |
| Example 1 | 97.6 | 87.4 | 0 | 80 | Normal |
| | | | | | pressure |
| Example 2 | 96.2 | 93.2 | 0 | 65 | Normal |
| | | | | | pressure |
| Example 3 | 98.5 | 90.5 | 0 | 80 | Normal |
| | | | | | pressure |
| Example 4 | 99.1 | 85.2 | 0 | 90 | Normal |
| | | | | | pressure |
| Example 5 | 97.8 | 88.7 | 0 | 80 | Normal |
| | | | | | pressure |
| Example 6 | 98.1 | 82.4 | 0 | 120 | Normal |
| | | | | | pressure |
| Example 7 | 95.7 | 93.5 | 0 | 80 | Normal |
| | | | | | pressure |
| Example 8 | 98.3 | 80.3 | 0 | 120 | Normal |
| | | | | | pressure |
| Example 9 | 94.4 | 93.2 | 0 | 80 | Normal |
| | | | | | pressure |
| Example 10 | 91.5 | 94.9 | 0 | 65 | Normal |
| | | | | | pressure |
| Example 11 | 96.9 | 91.8 | 0 | 80 | Normal |
| | | | | | pressure |
| Example 12 | 97.1 | 90.6 | 0 | 90 | Normal |
| | | | | | pressure |
| Example 13 | 94.5 | 92.1 | 0 | 80 | Normal |
| | | | | | pressure |
| Example 14 | 96.9 | 88.9 | 0 | 120 | Normal |
| | | | | | pressure |
| Example 15 | 93.8 | 94.1 | 0 | 80 | Normal |
| | | | | | pressure |
| Example 16 | 95.7 | 88.3 | 0 | 120 | Normal |
| | | | | | pressure |
| Example 17 | 95.3 | 80.3 | 0 | 80 | Normal |
| | | | | | pressure |
| Example 18 | 96.1 | 82.7 | 0 | 65 | Normal |
| | | | | | pressure |
| Example 19 | 98.2 | 84.8 | 0 | 80 | Normal |
| | | | | | pressure |
| Example 20 | 98.6 | 86.4 | 0 | 90 | Normal |
| | | | | | pressure |
| Example 21 | 98.1 | 88.2 | 0 | 80 | Normal |
| | | | | | pressure |
| Example 22 | 97.9 | 83.9 | 0 | 120 | Normal |
| | | | | | pressure |
| Example 23 | 94.7 | 84.2 | 0 | 80 | Normal |
| | | | | | pressure |
| Example 24 | 98.5 | 81.6 | 0 | 120 | Normal |
| | | | | | pressure |
| Example 25 | 97.9 | 84.6 | 0 | 80 | Normal |
| | | | | | pressure |
| Example 26 | 97.5 | 87.1 | 0 | 65 | Normal |
| | | | | | pressure |
| Example 27 | 98.4 | 88.3 | 0 | 80 | Normal |
| | | | | | pressure |
| Example 28 | 99.0 | 83.9 | 0 | 90 | Normal |
| | | | | | pressure |
| Example 29 | 98.1 | 89.7 | 0 | 80 | Normal |
| | | | | | pressure |
| Example 30 | 98.6 | 87.5 | 0 | 120 | Normal |
| | | | | | pressure |
| Example 31 | 96.3 | 85.6 | 0 | 80 | Normal |
| | | | | | pressure |
| Example 32 | 97.7 | 82.4 | 0 | 120 | Normal |
| | | | | | pressure |
| Comparative | 96.4 | 87.2 | 4 | 170 | 6.4 |
| Example 1 | | | | | |
| Comparative | 92.3 | 92.1 | 100 | 170 | 7.3 |
| Example 2 | | | | | |
| Comparative | 52.1 | 82.6 | 4 | 170 | 6.8 |
| Example 3 | | | | | |

In Examples 1 to 6, a deuterium substitution reaction was performed using each of dimethyl sulfoxide, tetrahydrofuran, 1,4-dioxane, methylcyclohexane, 1,2-dichloroethane or xylene as an organic solvent for 11,12-dihydro indolo[2,3-a]carbazole. In Examples 9 to 14, a deuterium substitution reaction was performed using each of dimethyl sulfoxide, tetrahydrofuran, 1,4-dioxane, methylcyclohexane, 1,2-dichloroethane or xylene as an organic solvent for carbazole. In Examples 17 to 22, a deuterium substitution reaction was performed using each of dimethyl sulfoxide, tetrahydrofuran, 1,4-dioxane, methylcyclohexane, 1,2-dichloroethane or xylene as an organic solvent for 2-bromo-dibenzofuran. In Examples 25 to 30, a deuterium substitution reaction was performed using each of dimethyl sulfoxide, tetrahydrofuran, 1,4-dioxane, methylcyclohexane, 1,2-dichloroethane or xylene as an organic solvent for 2-chloro-4,6-diphenyl-1,3,5-triazine.

In Examples 1, 7 and 8, a deuterium substitution reaction was performed by changing a compound which is hydrolyzed by heavy water for 11,12-dihydro indolo[2,3-a]carbazole into each of methanesulfonic anhydride, trifluoromethanesulfonic anhydride or trifluoroacetic anhydride. In Examples 9, 15 and 16, a deuterium substitution reaction was performed by changing a compound which is hydrolyzed by heavy water for carbazole into each of methanesulfonic anhydride, trifluoromethanesulfonic anhydride or trifluoroacetic anhydride. In Examples 17, 23 and 24, a deuterium substitution reaction was performed by changing a compound which is hydrolyzed by heavy water for 2-bromo-dibenzofuran into each of methanesulfonic anhydride, trifluoromethanesulfonic anhydride or trifluoroacetic anhydride. In Examples 25, 31 and 32, a deuterium substitution reaction was performed by changing a compound which is hydrolyzed by heavy water for 2-chloro-4,6-diphenyl-1,3,5-triazine into each of methanesulfonic anhydride, trifluoromethanesulfonic anhydride or trifluoroacetic anhydride.

The purity and deuterium substitution rate vary depending on the solubility of the reactant in the organic solvent and the solubility of the reactant in the heavy water that provides deuterium. For this reason, an organic solvent having good solubility in water is used. Further, as the amount of acid anhydride used increases, the solubility can be increased while increasing the acidity of the solution, to dissolve the reactant.

In Examples 1 to 32, carbazole having a high solubility in an organic solvent and a good affinity for heavy water resulted in a high deuterium substitution rate. The purity tends to be slightly contrary to the deuterium substitution rate, but the better the solubility in organic solvents and heavy water, the better the reactivity, and the more impurities due to side reactions. For this reason, carbazole tends to be less pure than other reactants.

Examples 1 to 32 were also performed under normal pressure without an increase in pressure during the reaction because the reaction was performed under acidic conditions. In Comparative Examples 1 to 3, deuterium substitution was performed in a high-pressure reactor using a catalyst, but a desired result may be obtained by performing deuterium substitution under normal pressure or more, that is, at least 5 bar or more. In addition, when deuterium substitution is performed using a high-pressure reactor, a side reaction occurs in which the double bond of an aromatic ring is partially reduced, but a side reactant thus formed is difficult to isolate, and even through the side reactant is isolated, the yield is significantly reduced.

Comparative Examples 1 and 2 are the results of comparing the changes in the deuterium substitution rate and the purity according to the proportion of the hydrogenated compound used when deuterium is substituted under high pressure using a catalyst. It can be seen that when the proportion of the hydrogenated compound is 4%, the purity is higher than when the proportion of the hydrogenated compound is 100%.

Examples 17 to 24 and Comparative Example 3 are experiments of comparing conditions under which deuterium is substituted using a compound which can be hydrolyzed by heavy water (Examples 17 to 24) with conditions under which deuterium is substituted under high pressure using a catalyst (Comparative Example 3), when a target compound has a halogen group which is a leaving group. This experiment is an experiment to confirm whether a halogen group, which is a leaving group after the deuterium substitution reaction, is well attached without being detached, and in Examples 17 to 24, a bromine group, which is a leaving group, was well attached even after the deuterium substitution reaction, but in Comparative Example 3, a peak due to dibenzofuran from which a bromine group, which is a leaving group, was partially detached was confirmed through HPLC.

1. A method for producing a deuterated aromatic compound, the method comprising: performing a deuterated reaction of an aromatic compound comprising one or more aromatic rings using a solution comprising the aromatic compound, heavy water, an organic compound which can be hydrolyzed by the heavy water, and an organic solvent.

2. The method of claim 1, wherein the organic solvent is a solvent comprising: a hydrocarbon chain which is unsubstituted or substituted with a halogen group; an aliphatic hydrocarbon ring which is unsubstituted or substituted with an alkyl group; an aromatic hydrocarbon ring which is unsubstituted or substituted with an alkyl group; a straight-chained or branched heterochain; a substituted or unsubstituted aliphatic hetero ring; or a substituted or unsubstituted aromatic hetero ring.

3. The method of claim 1, wherein the organic solvent comprises at least one of an oxygen element and a sulfur atom, and further comprises: a substituted or unsubstituted hetero ring; a substituted or unsubstituted alkyl acetate; alkyl ketone; alkyl sulfoxide; a lactone having 4 to 10 carbon atoms; alkylamide; a glycol having 4 to 10 carbon atoms; dioxane; or an acetic acid which is unsubstituted or substituted with alkoxy.

4. The method of claim 1, wherein the organic solvent comprises: ethyl acetate, acetone, cyclohexanone, methyl ethyl ketone, tetrahydrofuran, tetrahydropyran, cyclopentanone, 1,2-dioxane, 1,3-dioxane, 1,4-dioxane, N,N-dimethylformamide, dimethyl sulfoxide, 1,2-dimethoxyethane, diglyme, γ-butyrolactone, γ-valerolactone, methyl ethyl diglycol, propylene glycol methyl ether, propylene glycol methyl ether acetate, ethyl lactate, cyclohexane, methylcyclohexane, ethylcyclohexane, diethyl ether, decalin, hexane, heptane, toluene, xylene, 1,3,5-trimethylbenzene, dichloromethane, 1,2-di chloroethane, 1,1,2,2-tetrachloroethane, tetrachloroethylene or 2-methoxyacetic acid.

5. The method of claim 1, wherein the organic compound which can be hydrolyzed by the heavy water comprises at least one compound of the following Chemical Formulae 1 to 4:
R1-C(O)OC(O)—R2 [Chemical Formula 1]
R3-S(O₂)OS(O₂)—R4 [Chemical Formula 2]
R5-C(O)O—R6 [Chemical Formula 3]
R7-CONH—R8 [Chemical Formula 4]
in Chemical Formulae 1 to 4,
R1 to R8 are the same as or different from each other, and are each independently a monovalent organic group.

6. The method of claim 1, wherein the organic compound which can be hydrolyzed by the heavy water comprises at least one of trifluoromethanesulfonic anhydride, trifluoroacetic anhydride, acetic anhydride and methanesulfonic anhydride.

7. The method of claim 1, wherein performing of the deuterated reaction comprises:
preparing a solution comprising an aromatic compound comprising one or more aromatic rings, heavy water, an organic compound which can be hydrolyzed by the heavy water, and an organic solvent; and
performing the deuterated reaction of the aromatic compound by heating the solution.

8. A deuterated reaction composition comprising an aromatic compound comprising one or more aromatic rings, heavy water (D₂O), an organic compound which can be hydrolyzed by the heavy water, and an organic solvent.

9. The deuterated reaction composition of claim 8, wherein the organic solvent is a solvent comprising: a hydrocarbon chain which is unsubstituted or substituted with a halogen group; an aliphatic hydrocarbon ring which is unsubstituted or substituted with an alkyl group; an aromatic hydrocarbon ring which is unsubstituted or substituted with an alkyl group; a straight-chained or branched heterochain; a substituted or unsubstituted aliphatic hetero ring; or a substituted or unsubstituted aromatic hetero ring.

10. The deuterated reaction composition of claim 8, wherein the organic solvent comprises at least one of an oxygen element and a sulfur atom, and further comprises: a substituted or unsubstituted hetero ring; a substituted or unsubstituted alkyl acetate; alkyl ketone; alkyl sulfoxide; a lactone having 4 to 10 carbon atoms; alkylamide; a glycol having 4 to 10 carbon atoms; dioxane; or an acetic acid which is unsubstituted or substituted with alkoxy.

11. The deuterated reaction composition of claim 8, wherein the organic solvent comprises: ethyl acetate, acetone, cyclohexanone, methyl ethyl ketone, tetrahydrofuran, tetrahydropyran, cyclopentanone, 1,2-dioxane, 1,3-dioxane, 1,4-dioxane, N,N-dimethylformamide, dimethyl sulfoxide, 1,2-dimethoxyethane, diglyme, γ-butyrolactone, γ-valerolactone, methyl ethyl diglycol, propylene glycol methyl ether, propylene glycol methyl ether acetate, ethyl lactate, cyclohexane, methylcyclohexane, ethylcyclohexane, diethyl ether, decalin, hexane, heptane, toluene, xylene, 1,3,5-trimethylbenzene, dichloromethane, 1,2-di chloroethane, 1,1,2,2-tetrachloroethane, tetrachloroethylene and or 2-methoxyacetic acid.

12. The deuterated reaction composition of claim 8, wherein the organic compound which can be hydrolyzed by the heavy water comprises at least one compound of the following Chemical Formulae 1 to 4:
R1-C(O)OC(O)—R2 [Chemical Formula 1]
R3-S(O₂)OS(O₂)—R4 [Chemical Formula 2]
R5-C(O)O—R6 [Chemical Formula 3]
R7-CONH—R8 [Chemical Formula 4]
in Chemical Formulae 1 to 4,
R1 to R8 are the same as or different from each other, and are each independently a monovalent organic group.

13. The deuterated reaction composition of claim 8, wherein the organic compound which can be hydrolyzed by the heavy water comprises at least one of trifluoromethanesulfonic anhydride, trifluoroacetic anhydride, acetic anhydride and methanesulfonic anhydride.

14. A deuterated aromatic compound produced by the method of claim 1.

15. The deuterated aromatic compound of claim 14, wherein the deuterated aromatic compound comprises a substituent comprising: a leaving group, a hydroxyl group, a substituted or unsubstituted amine group and a cyano group.

16. The deuterated aromatic compound of claim 15, wherein the leaving group is selected from the group comprising a halogen group or a boronic acid group.

17. The deuterated aromatic compound of claim 15, wherein the deuterated aromatic compound comprising a leaving group, a hydroxyl group, a substituted or unsubstituted amine group and a cyano group is any one of the following Chemical Formulae 7 to 10: 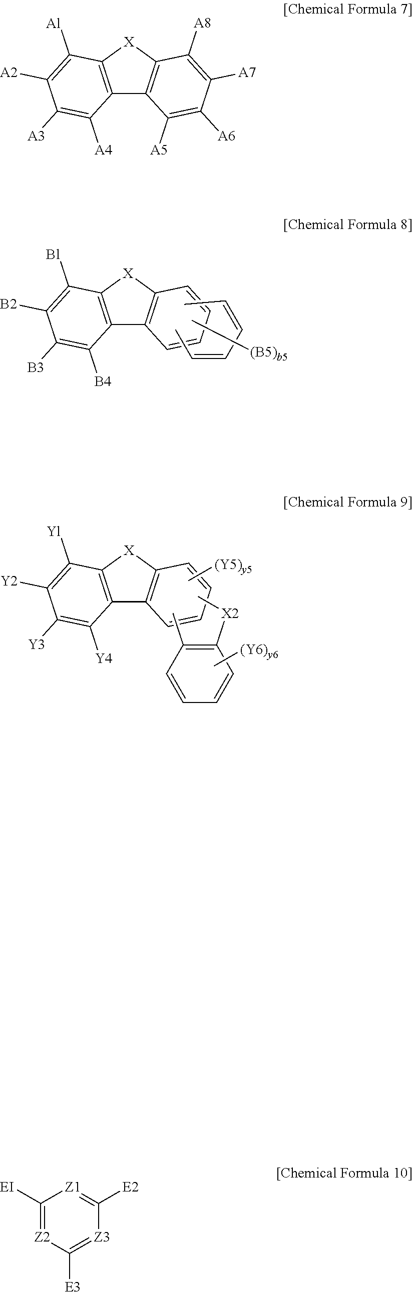
in Chemical Formulae 7 to 10,
X, X1 and X2 are each independently 0, S or NR, wherein
R is hydrogen; deuterium; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,
at least one of A1 to A8 is deuterium, at least one is a substituent comprising a leaving group, a hydroxyl group, a substituted or unsubstituted amine group or a cyano group, and the others are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,
at least one of B1 to B5 is deuterium, at least one is a substituent comprising a leaving group, a hydroxyl group, a substituted or unsubstituted amine group and a cyano group, and the others are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,
at least one of E1 to E3 is an aryl group which is substituted with deuterium; or a heterocyclic group which is substituted with deuterium, at least one is a substituent comprising a leaving group, a hydroxyl group, a substituted or unsubstituted amine group and a cyano group, and the others are each independently hydrogen; a leaving group; a substituted or unsubstituted alkyl group; a substituted or unsubstituted cycloalkyl group; a substituted or unsubstituted aryl group; or a substituted or unsubstituted heterocyclic group,
at least one of Y1 to Y6 is deuterium, at least one is a substituent comprising a leaving group, a hydroxyl group, a substituted or unsubstituted amine group and a cyano group, and the others are each independently hydrogen; a leaving group; a hydroxyl group; a substituted or unsubstituted amine group; a cyano group; a substituted or unsubstituted alkyl group; a substituted or unsubstituted cycloalkyl group; a substituted or unsubstituted aryl group; or a substituted or unsubstituted heterocyclic group,
at least one of Z1 to Z3 is N, and the others are each independently CH or N,
b5 is an integer from 1 to 6, and when b5 is 2 or higher, B5's are the same as or different from each other,
y5 is 1 or 2, and when y5 is 2, Y5's are the same as or different from each other, and
y6 is an integer from 1 to 4, and when y6 is 2 or higher, Y6's are the same as or different from each other.

18. The deuterated aromatic compound of claim 15, wherein the deuterated aromatic compound comprising the substituent comprises any one of the following structures, and the structures are each substituted with one or more deuteriums: 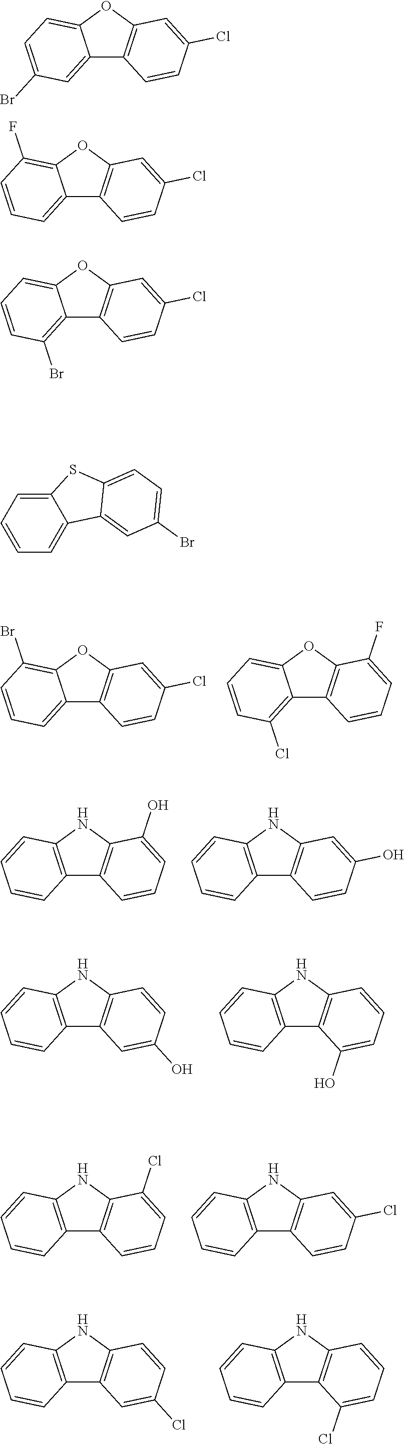 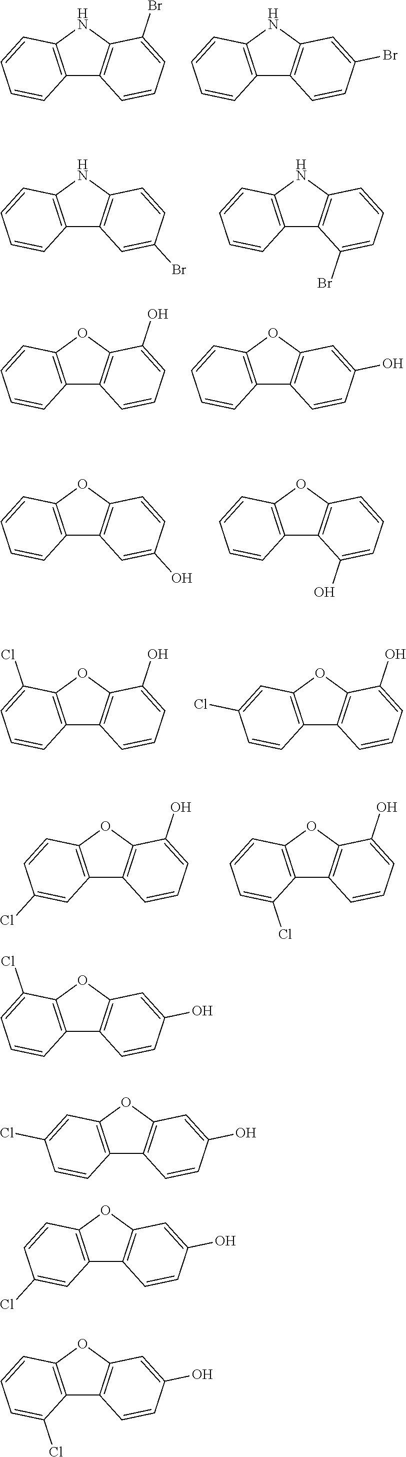 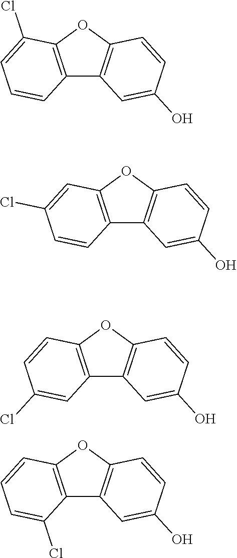 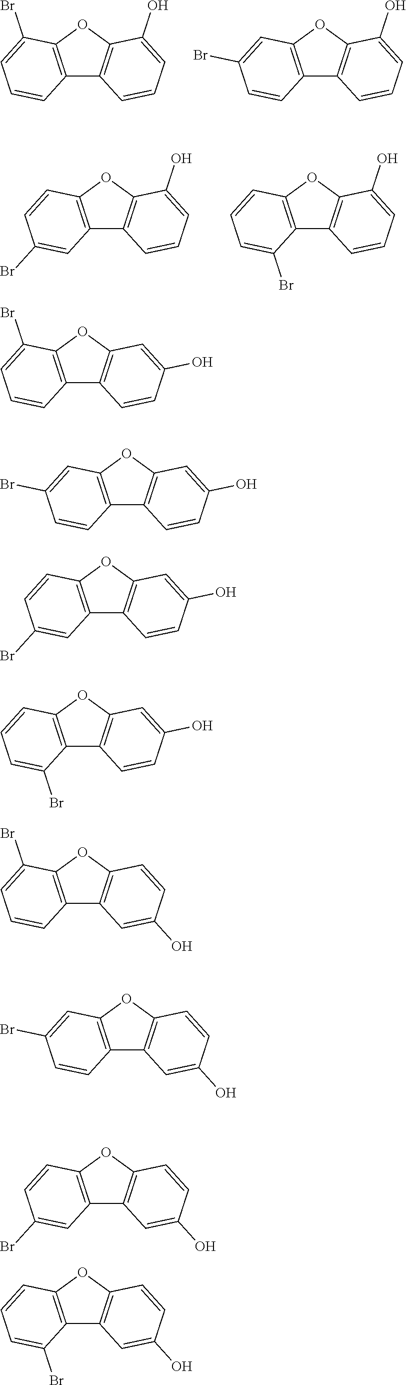 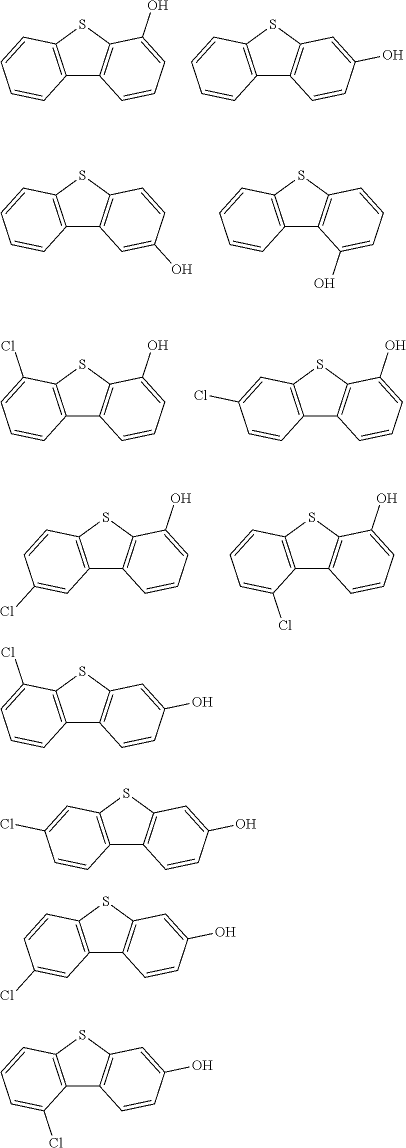 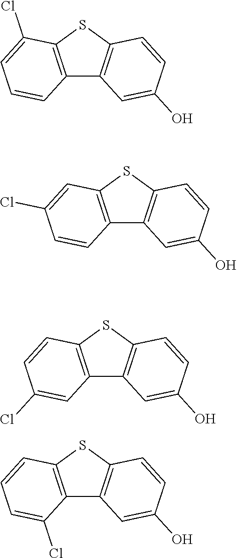 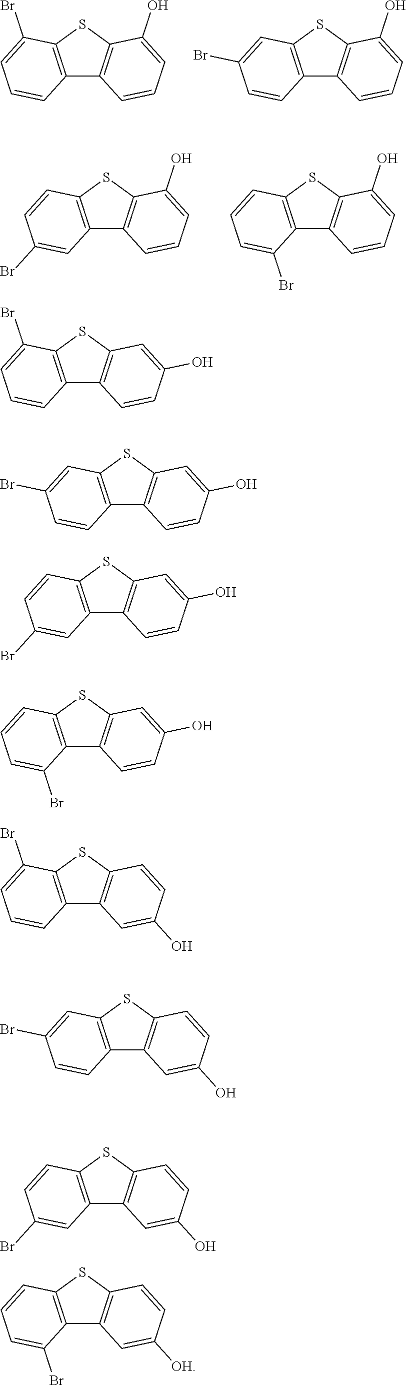 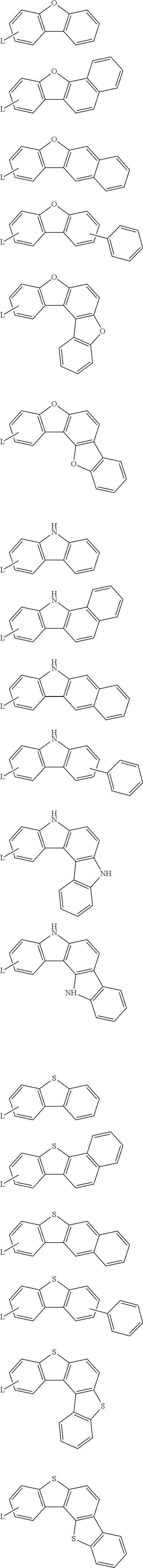 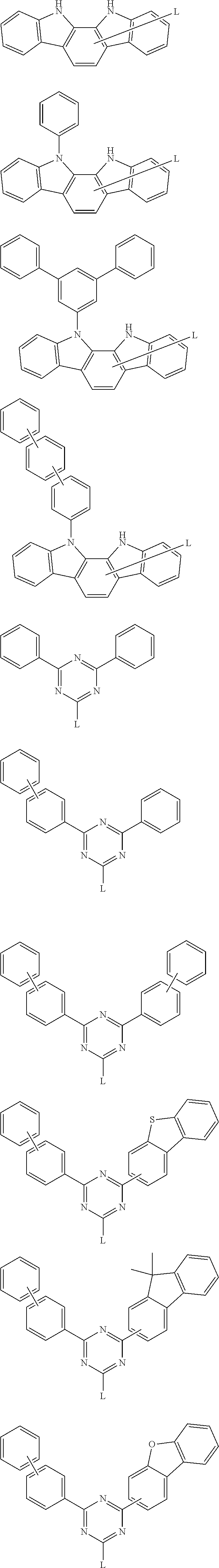
wherein, L is a leaving group, a hydroxyl group, a substituted or unsubstituted amine group, or a cyano group.

19. An electronic device comprising the deuterated aromatic compound of claim 14.
